(19) United States
(12) Patent Application Publication
(10) Pub. No.: US 2013/0072899 A1
(43) Pub. Date: Mar. 21, 2013

(54) Pharmaceutical Formulations for Iontophoretic Delivery of Gallium

(71) Applicant: Nitric BloTherapeutics, Inc., Bristol, PA (US)

(72) Inventors: Phillip M. Friden, Bedford, MA (US); Hyun D. Kim, Weston, MA (US); Kirsten Staff, Parsons Green (GB); Marc B. Brown, Watford (GB)

(73) Assignee: Nitric BloTherapeutics, Inc., Bristol, PA (US)

(21) Appl. No.: 13/679,579

(22) Filed: Nov. 16, 2012

Related U.S. Application Data
(63) Continuation of application No. 13/426,369, filed on Mar. 21, 2012.
Application No. 13/426,369 is a continuation of application No. 13/207,195, filed on Aug. 10, 2011, now abandoned.
Application No. 13/207,195 is a continuation of application No. 12/999,279, now abandoned.
(60) Provisional application No. 61/073,158, filed on Jun. 17, 2008.

(51) Int. Cl. A61K 33/24; A61N 1/30

(52) U.S. Cl. CPC A61K 33/24; A61N 1/30
USPC 604/501; 424/650

(57) ABSTRACT

Pharmaceutical formulations suitable for iontophoresis thereof that provide enhanced iontophoretic delivery of gallium to at least one body surface are described and methods for administering gallium to a body surface via iontophoresis. In one embodiment, the body surface is human skin.

### RELATED APPLICATION

This application claims the benefit of Provisional Application No. 61/073,158 filed on Jun. 17, 2008. The entire teachings of the above application are incorporated herein by reference.

### BACKGROUND OF THE INVENTION

An iontophoretic delivery system is, for example, a drug delivery system that releases drug at a controlled rate to the target tissue upon application. The advantages of systems wherein drug is delivered locally via iontophoresis are the ease of use, being relatively safe, and affording the interruption of the medication by simply stopping the current and/or peeling off or removing it from the skin or other body surface whenever an overdosing is suspected. The total skin surface area of an adult is about 2 m². In recent years, iontophoretic delivery of drugs has attracted wide attention as a better way of administering drugs for local as well as systemic effects. The design of iontophoretic delivery systems can usually be such that the side effects generally seen with the systmeic administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions through the epidermal or dermal layers into a target tissue. Particularly suitable target tissues include those adjacent to the delivery site for localized treatment. Unchanged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode.

U.S. Pat. No. 6,477,410 issued to Henley et al. describes the use of iontophoresis for drug delivery. However, there remains a need for improved formulations that facilitate the delivery of specific active agents.

Gallium is a group III metal which has been described as useful in the treatment of hypercalcemia of malignancy, osteoporosis and cancer. Gallium has additionally been demonstrated to upregulate collagen and fibronectin synthesis in the skin (Bockman et al, (1993). J Cell Biochem. 52(4):396-403). In order for gallium to be an effective drug used in the treatment of certain diseases and conditions of the skin, it must permeate through the skin in a sufficient quantity. The ability of many drugs, including gallium, to passively diffuse into the skin is limited because the drug must be formulated to have adequate aqueous and lipid solubility to diffuse through the different layers of skin. Therefore, there remains a need in the art for improved methods of delivering gallium into the skin.

### SUMMARY OF THE INVENTION

The present invention provides pharmaceutical formulations suitable for iontophoresis that provide enhanced iontophoretic delivery of gallium to at least one body surface.

In one embodiment, the formulation comprises gallium nitrate in a buffer, wherein the buffer has an ionic strength and pH suitable for the formation of a charged gallium species. In one embodiment the gallium species is selected from the group consisting of gallium hydroxide and gallium citrate species.

In another embodiment, the formulation comprises gallium nitrate in a buffer having an ionic strength from about 0.01 to about 1 M.

In a further embodiment, the formulation comprises gallium nitrate in a buffer and has a pH from about 2 to about 12. In yet another embodiment, the formulation has a pH from about 2 to about 10.

In an additional embodiments the formulation comprises gallium nitrate in a buffer and has a pH from about 3 to about 4.

In another embodiment, the formulation comprises gallium nitrate in a buffer and has a pH from about 7 to about 8.

In certain embodiments, the formulation comprises gallium nitrate in a buffer, wherein the buffer is a citrate buffer.

In yet another embodiment, the formulation comprises gallium nitrate in a citrate buffer wherein the gallium species in the formulation comprises gallium citrate species.

In a further embodiment, the formulation comprises gallium nitrate in a citrate buffer wherein the gallium species in the formulation comprises gallium hydroxide species.

In an additional embodiment, the invention is directed to a method for administering gallium to a patient in need thereof comprising iontophoretically administering to a body surface of the patient a formulation of the invention.

In yet another embodiment, the invention is directed to a method of increasing the production of collagen or fibronectin in a patient in need thereof comprising iontophoretically administering to the body surface of the patient. In one embodiment, the body surface is the skin. In another embodiment, the invention is directed to a method of increasing skin thickness and/or elasticity.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a graph that shows the percent of gallium recovery (% recovery) as a measure of stability in a 50 mM pyrrolidine buffer over 100 hours at 0.5 mg/ml gallium (♦), 0.25 mg/ml gallium (▪) and 0.125 mg/ml gallium (▴) (n=3, ±1 s.d.).

FIG. 2 is a graph that shows the percent of gallium recovery (% recovery) as a measure of stability in a 50 mM citric acid buffer over 100 hours at 0.5 mg/ml gallium (♦), 0.25 mg/ml gallium (▪) and 0.125 mg/ml gallium (▴) (n=3, ±1 s.d.).

FIG. 3 is a graph shows the percent of gallium recovery (% recovery) as a measure of stability in a 50 mM formic acid buffer over 100 hours at 0.5 mg/ml gallium (♦), 0.25 mg/ml gallium (▪) and 0.125 mg/ml gallium (▴) (n=3, ±1 s.d.).

FIG. 4 is a graph that shows the permeation of gallium (mg/cm²) info the epidermal sheet by passive permeation or iontophoresis over 24 hours.

FIG. 5A is a graph that shows the permeation of gallium (mg/cm²) through full thickeness human skin by iontophoresis (400 μA for 10 min) over 70 hours using three concentrations of gallium nitrate (saturated 16.67% w/v ▴, 1.5% w/v ♦ or 0.15% w/v ▪) in 50 mM citric acid buffer (pH 5.0).

FIG. 5B is a graph that shows the permeation of gallium (mg/cm²) through full thickeness human skin by passive permeation (♦) or iontophoresis (400 μA for 10 min ▪ or 60 min ▴) over 70 hours using 16.67% w/v (saturated) gallium nitrate solution in 50 mM citric acid buffer (pH 5.0).

FIG. 6 is a graph comparing tritiated water (3.7 Bq/mL) permeation across full thickness human skin using upright Franz cells (n=4, ±s.d.) and 0.5 M citric acid buffer at pH 2 vs. pH 5 (▴=pH 2, ▪=pH 5).

FIGS. 7A, 7B and 7C are HYSS speciation plots showing the effect on gallium speciation of increasing gallium concentration (a=0.15, b=1.5, c=16.67% w/v Ga) in a 0.05 M citrate buffer solution between pH range 0-10. Where Ga=free gallium ions, GaL=gallium citrate complex, GaH₋₃=Ga(OH)₃, GaH₋₂=Ga(OH)₂¹⁺, GaH₋₁=Ga(OH)²⁺ and GaH₋₄=Ga(OH)₄⁻. Plot A shows the composition of GACIT100 formulation, plot B shows the composition of GAOHMIXB formulation and plot C shows GAOHMIXC composition (GACIT100, GAOHMIXB and GAOMIXC formulations are described in Example 5).

FIGS. 8A, 8B and 8C are HYSS speciation plots showing the effect on gallium speciation of decreasing buffer strength (a=1, b=0.1, c=0.05 M citrate buffer) in a saturated gallium solution of 16.67% w/v between pH range 0-10. Where Ga=free gallium ions, GaL=gallium citrate complex, GaH₋₃=Ga(OH)₃, GaH₋₂=Ga(OH)₂¹⁺, GaH₋₁=Ga(OH)²⁺ and GaH₋₄=Ga(OH)₄⁻. Plot A shows the composition of GACIT50 formulation and plot B shows the composition of GAOHMIXA formulation (GACIT50 and GAOHMIXA formulation are described in Example 5).

FIG. 9A is a graph that shows gallium skin deposition (ng gallium per mg tissue) of 0.15% w/v gallium nitrate in 0.05M citrate butler at pH 2 after passive, anodal, and cathodal iontophoresis at 0.3 mA/cm² for 15 min.

FIG. 9B is a graph that shows gallium skin deposition (ng gallium per mg tissue) of 0.15% w/v gallium nitrate in 0.05M citrate buffer at pH 2, 3, 4, and 6 after cathodal iontophoresis at 0.3 mA/cm² for 15 min.

FIG. 10A is a graph that shows gallium skin deposition (ng gallium per mg tissue) of 16.6% w/v gallium nitrate in 1M citrate buffer at pH 6 after passive, anodal, and cathodal iontophoresis at 0.3 mA/cm² for 15 min.

FIG. 10B is a graph that shows gallium skin deposition (ng gallium per mg tissue) of 16.6% w/v gallium nitrate in 1M citrate buffer at pH 2, 4, 6, 7, and 8 after cathodal iontophoresis at 0.3 mA/cm² for 15 min.

### DETAILED DESCRIPTION OF THE INVENTION

The invention is directed to pharmaceutical formulations of gallium that are suitable for iontophoresis, methods of administering gallium to a body surface of a patient in need thereof and methods of increasing the production of collagen or fibronectin in a patient in need thereof comprising iontophoretically administering a formulation of the invention to a body surface of said patient. In one embodiment, the body surface is the skin.

In one embodiment, the invention is directed to a formulation suitable for iontophoretic delivery of gallium wherein the formulation comprises a gallium-containing compound. In a further embodiment, the gallium containing compound or species formed in solution is such that the gallium compound(s) (or species) is soluble and possesses a net charge.

Gallium-containing compounds include, but are not limited to, gallium nitrate, gallium phosphate, gallium citrate, gallium chloride, gallium fluoride, gallium carbonate, gallium formate, gallium acetate, gallium tartrate, gallium maltol, gallium oxalate, gallium oxide, hydrated gallium oxide, peptide-bound gallium and pre-complexed coordination complex of gallium. In yet another embodiment, the invention is a method of administering gallium to a patient in need thereof comprising iontophoretically administering a gallium-containing compound.

In certain embodiments, the invention is directed to a formulation comprising gallium nitrate in a buffer wherein the gallium species comprises a charged species. In another embodiment, the formulation comprises gallium citrate or gallium hydroxide species.

In one embodiment, the buffer has an ionic strength from about 0.01 M to about 1 M. In certain embodiments, the buffer has an ionic strength from about 0.05 M to about 0.10 M.

In another embodiment, the buffer is selected from the group consisting of a citrate buffer, formic acid buffer, borate buffer and a pyrrolidone buffer. In another embodiment the buffer is a citrate buffer. In yet another embodiment, the buffer has a pKa from about 2 to about 12.

In a further embodiment, the formulation comprises gallium nitrate in other carboxylic acid buffers such as tartrate, malate, fumarate, edetate, gluconate, succinate, phosphate, and amino acids.

In certain embodiments, the formulation comprises gallium nitrate in a buffer wherein the pH of the formulation is from about 2 to about 12. In a further embodiment, the pH of the formulation is from about about 2 to about 10. In another embodiment, the pH of the formulation is from about 3 to about 4. In an additional embodiment, the pH of the formulation is from about 6 to about 10. In a further embodiment, the pH of the formulation is from about 7 to about 8.

In an additional embodiment, the invention is directed to a formulation suitable for iontophoresis comprising gallium nitrate in a buffer wherein the buffer has an ionic strength from about 0.01 M to about 1 M. In another embodiment, the formulation has a pH from about 3 to about 6.

In other embodiments, the formulation comprises gallium nitrate in a buffer wherein the buffer is a citrate buffer at an ionic strength from about 0.01 M to about 1 M and wherein the formulation has a pH from about 2 to about 12. In an additional embodiment, the ionic strength is from about 0.05 to about 0.10 M. In yet another embodiment, the pH is from about 2 to about 10.

In addition to the gallium-containing compound (such as gallium nitrate) and the buffer, the formulation can contain an additional pharmaceutically acceptable carrier or excipient. As used herein, the term “pharmaceutically acceptable carrier or excipient” means any non-toxic diluent or other formulation auxiliary that is suitable for use in iontophoresis. Examples of pharmaceutically acceptable earners or excipients include but are not limited to: diluents such as water, or other solvents, cosolvents; solubilizing agents such as sorbital and glycerin; pharmaceutically acceptable bases; viscosity modulating agents such as cellulose and its derivatives; permeation enhancers; and stabilizers.

The formulation comprises the gallium containing compound in a therapeutically effective amount. In one embodiment, the formulation comprises gallium nitrate in a therapeutically effective amount. A “therapeutically effective amount” is an amount of gallium containing compound that is sufficient to prevent development of or to alleviate to some extent one or more of a patient's symptoms of the disease or condition being treated. In certain embodiments, the formulation comprises gallium nitrate in an amount sufficient to increase the production of collagen and/or increase the production of fibronectin in the skin and/or promote wound healing and/or reduce wrinkles in the skin and/or reduce photodamage.

In one embodiment, the concentration of gallium nitrate in the formulation is from about 0.1 to about 20% w/v gallium nitrate. In other embodiments, the concentration of gallium nitrate is from about 15% (w/v) to about 20% (w/v). In yet another embodiment, the concentration of gallium nitrate is from about 16 to about 17% (w/v). In an additional embodiment, the concentration of gallium nitrate is about 16.7% (w/v).

In an additional embodiment, the concentration of gallium nitrate is from about 0.1 to about 2% (w/v). In another embodiment, the concentration of gallium nitrate is from about 0.5 to about 1.5% (w/v). In further embodiments, the concentration of gallium nitrate is from about 0.1 to about 0.2% (w/v). In a particular embodiment, the concentration of gallium nitrate is about 0.15% (w/v).

In solution, gallium nitrate forms several gallium species including free cations and coordination complexes. For example, citrate buffered solutions of gallium nitrate comprise free gallium cations (Ga³⁺) as well as coordination complexes. These gallium coordination complexes include gallium citrate and gallium hydroxide species. Gallium hydroxide species include GaOH²⁺, Ga(OH)₂⁺, Ga(OH)₃ and Ga(OH)₄⁻. The term “positively charged gallium hydroxide species” is meant to encompass GaOH²⁺ and Ga(OH)₂⁺. The gallium species that are formed in formulations comprising gallium in a citrate buffer is dependent on several factors, including the concentration of gallium, buffer strength and pH. In one embodiment of the present invention, the gallium species in the formulation comprise gallium citrate. In another aspect of the invention, the gallium species in the formulation consists of gallium citrate. In another embodiment, the gallium species in the formulation comprise gallium hydroxide species. In yet another embodiment, the gallium species in the formulation comprises positively charged gallium hydroxide species. In certain other aspects, the formulation comprises gallium citrate and gallium hydroxide species. In another aspect, the formulation comprises gallium citrate and positively charged gallium hydroxide species. In yet other aspects, the formulation consists of free gallium, gallium citrate and gallium hydroxide species selected from the group consisting of Ga(OH)₂⁺, Ga(OH)²⁺ and Ga(OH)₃.

In some embodiments, the formulation is suitable for anodal iontophoresis. A formulation is suitable for anodal iontophoresis when it comprises positively charged gallium species, such as positively charged gallium hydroxide species.

In other embodiments, the formulation is suitable for cathodal iontophoresis. A formulation is suitable for cathodal iontophoresis when it comprises negatively charged gallium species, such as gallium citrate species.

In other embodiments, the invention is a formulation wherein the iontophoretic delivery of gallium species results in increased permeation through the skin and/or deposition in the skin compared to passive delivery of the gallium species. In one embodiment, iontophoretic delivery of gallium species results in at least about a 50% increase in permeation or deposition compared to passive delivery. In another embodiment, the iontophoretic delivery of gallium species results in at least at least about a 100% increase compared to passive delivery. In a further embodiment, the iontophoretic delivery of gallium species results in at least about a 500% increase compared to passive delivery. In another embodiment, the iontophoretic delivery of gallium species results in at least about a 1,000% increase compared to passive delivery. In yet another embodiment, the iontophoretic delivery of gallium species results in at least about a 10,000% increase compared to passive delivery.

In some embodiments, the formulation comprises gallium nitrate in a citrate buffer wherein the buffer has an ionic strength of about 1 M, a pH from about 6 to about 10 and gallium nitrate at a concentration of about 16.7% w/v.

In other embodiments, the formulation comprises gallium nitrate in a citrate buffer wherein the buffer has an ionic strength of about 1 M, a pH from about 7 to about 8 and gallium nitrate at a concentration of about 16.7% w/v.

In another embodiment of the invention, the formulation comprises gallium nitrate in a citrate buffer wherein the buffer has an ionic strength of about 0.05 M, a pH of about 4 and gallium nitrate at a concentration of about 16.7% w/v.

In yet another embodiment, the formulation comprises gallium nitrate in a citrate buffer wherein the buffer has an ionic strength of about 0.10 M, a pH of about 3.8 and gallium nitrate at a concentration of about 16.7% w/v.

In a further embodiment, the formulation comprises gallium nitrate in a citrate buffer wherein the buffer has an ionic strength of about 0.05 M, a pH of about 4.1 and gallium nitrate at a concentration of about 0.15% w/v.

In an additional embodiment, the formulation comprises gallium nitrate in a citrate buffer wherein the buffer has an ionic strength of about 1.0 M, a pH of about 5.2 and gallium nitrate at a concentration of about 16.7% w/v.

In yet another embodiment, the formulation comprises gallium nitrate in a citrate buffer wherein the buffer has an ionic strength of about 1.0M, a pH of about 7.4 and gallium nitrate at a concentration of about 16.7%.

In an additional embodiment, the formulation comprises gallium nitrate in a citrate buffer wherein the buffer has an ionic strength of about 0.5 M, a pH of about 2 and gallium nitrate at a concentration of about 0.15% gallium nitrate.

In a further embodiment, the invention is a formulation suitable for iontophoresis comprising a pre-complexed coordination complex of gallium. A pre-complexed coordination complex is a gallium containing compound that forms a stable species with a small molecule. Such stable species with small molecules include gallium maltolate, gallium quinolinolonate, gallium thiosemicarbazone complexes, gallium hydroxypyridinone complexes, gallium hydroxypyrone complexes, gallium alkylcarboxylato complexes, gallium hydroxyaryl complexes, gallium tetra- or penta-methylene complexes, and gallium hydroxyquinoline complexes. In another embodiment, the pre-complexed coordination complex forms a stable species with a chelating agent. In a further embodiment, the pre-completed coordination complex forms a stable species with a small molecule selected from the group consisting of a catecholate, a hydroxamate, a pyridinone, and a pyridoxyl isonicotinoyl hydrazone.

In an additional embodiment, the gallium-containing compound is peptide-bound gallium. The term “peptide” expressly encompasses proteins and antibodies. Exemplary proteins include transferrin, lactoferrin, apolactoferrin and ferritin or fragments thereof that are capable of binding gallium.

The invention also encompasses methods of administering gallium to a patient in need thereof comprising iontophoretically administering to the skin of the patient a formulation comprising gallium nitrate.

In other embodiments, the invention is to a method of administering gallium comprising iontophoretically administering to the skin of the patient a formulation of the invention.

The formulations and methods of the present invention are useful for stimulating collagen or fibronectin synthesis in the skin. Increased stimulation of collagen and/or fibronectin synthesis has many therapeutic utilities including, for example, reducing photodamage to the skin caused by UV light exposure, increasing skin thickness and elasticity and promoting wound healing.

The inventive methods and formulation can be utilized for cosmetic purposes to decrease the signs of aging or photodamage by increasing skin thickness and/or elasticity. In one embodiment, the increased skin thickness and/or elasticity results in a reduction in wrinkles.

In addition, several adrenal and pituitary disorders, such as Cushing's disease, are associated with skin thinning and weakness. Skin atrophy is also an adverse effect associated with the long term use of corticosteroids. Therefore, in some embodiments, the inventive formulation or methods are used for increasing the thickness of skin in a patient suffering from an adrenal or pituitary disorder or those being administered a corticosteroid.

A wound is an injury characterized by an opening or breaking of the skin. Wound healing involves repair and regeneration of the skin tissue and occurs in three stages: inflammation, proliferation and maturation. Collagen production is necessary for the proliferation and maturation stages of wound healing. As used herein, promotion of wound healing includes accelerating or enhancing wound healing. In one embodiment, a method of the invention accelerates wound healing by at least about 5% as compared to wound healing in the absence of iontophoretic administration of gallium nitrate. In other embodiments, wound healing is accelerated by at least about 7%, 10%, 15%, 20%, 25%, 30%, 35%, 40%, 45%, 50%, 55%, 60%, 65%, 70%, 75%, 80%, 85%, 90%, 95%, 98% or 99%.

The formulations and methods of the invention also have utility in the treatment of hypercalcemia of malignancy, osteoporosis, osteoarthritis and cancer. “Treating” or “treatment” includes preventing or delaying the onset of the symptoms, complications, or biochemical indicia of a disease, alleviating or ameliorating the symptoms or arresting or inhibiting further development of the disease, condition, or disorder.

The current density that is applied is a current density that is sufficient for the gallium species to permeate the skin. In one embodiment, a current density of at least about 10 uA/cm² is applied. In another embodiment, a current density of at least about 50 uA/cm² is applied. In yet another embodiment, a current density of at least about 100 uA/cm² is applied. In another embodiment, a current density of at least about 200 uA/cm² is applied. In an additional embodiment, a current density of at least about 400 uA/cm² is applied. In yet another embodiment, a current density of at least about 500 uA/cm² is applied. In a further embodiment, a current density of at least about 600 uA/cm² is applied. In additional embodiments, a current density from about 200 uA/cm² to about 500 uA cm² is applied.

The time over which current density is applied is any time sufficient for the gallium species to permeate the skin. In one embodiment, the current is applied for a time greater than about 5 minutes. In another embodiment, the current is applied for a time greater than about 10 minutes. In yet another embodiment, the current is applied for a time greater than about 15 minutes. In a further embodiment, the current is applied for a time greater than about 30 minutes. In another embodiment, the current is applied for a time greater than about 60 minutes. In yet another embodiment, the current is applied for a time greater than about 2 hours. In an additional embodiment, the current is applied for about 10 minutes. In yet another embodiment, the current is applied for about 60 minutes. In a further embodiment, the current is applied for about 2 hours. In another embodiment, the current is applied for about 4 hours. In a further embodiment, the current is applied for about 8 hours. In one embodiment, the current is applied overnight.

In an additional aspect, iontophoresis is applied over a time and using a current that is sufficient for the gallium species to permeate the skin. In one embodiment, the current applied and the time over which it is applied result in Coulombic dose of about 0.1 to about 100 mA*min. In a further embodiment, the Coulombic dose is about 0.1 mA*min to about 10 mA*min.

In another embodiment, the inventive formulation is iontophoretically administered to a body surface once. In another embodiment, the inventive formulation is iontophoretically administered to the body surface at least twice. In a further embodiment, the inventive formulation can be iontophoretically administered to the body surface at least three times. In a further embodiment, the inventive formulation is iontophoretically administered to the body surface at least one time per week. In another embodiment, the inventive formulation is iontophoretically administered at an interval from once a week to once every four weeks. In another embodiment, the formulation is administered to the body surface at an interval from once every two weeks to once every four weeks. In a further embodiment, the formulation is administered to the body surface at an interval from once every three weeks to once every four weeks.

The formulation comprising gallium nitrate can be administered using an iontophoretic delivery device. In one embodiment, the formulation is adsorbed onto a flexible foam or pad or into a gel or as a film and applied to the body surface. In a further embodiment, the formulation can be administered using a drug cartridge pad. Such drug cartridges have been described in U.S. Pat. Nos. 6,148,231, 6,385,487, 6,477,410, 6,553,253, and U.S. Patent Publication Numbers 2004/0111051, 2003/0199808,2004/0039328, 2002/0161324, all incorporated herein by reference. In yet another embodiment, the formulation is preloaded into the applicator and distributed as a single use, single dose applicator for administration using an iontophoretic delivery device. Examples of iontophoretic delivery devices useful with the compositions and methods of the invention include, but are not limited to, handheld devices and devices which comprise a separate compartment as a power supply. Exemplary devices include, but are not limited to, those described in U.S. Pat. Nos. 6,148,231, 6,385,487, 6,477,410, 6,553,253, and U.S. Patent Publication Nos. 2004/0111051, 2003/0199808, 2004/0039328, 2002/0161324, and U.S. application Ser. No. 60/743,528, all incorporated herein by reference. An example of an applicator which can be used with a formulation of the invention comprises an active electrode adhered to an open cell polymer foam or hydrogel. Another applicator which has been developed for use with a device for iontophoretic delivery of an agent to a treatment site comprises an applicator head having opposite faces and including an active electrode and a porous pad (such as a woven or non-woven polymer, for example, a polypropylene pad); a margin of the applicator head about the active electrode having a plurality of spaced projections there along; the porous pad and the applicator head being ultrasonically welded to one another about the margin of the head with the electrode underlying the porous pad; and a medicament or a medicament and an electrically conductive earner therefor earned by the porous pad in electrical contact with the electrode. In one embodiment, the formulation is iontophoretically administered using carbon electrodes, silver-silver chloride electrodes or silver coated carbon electrodes.

The following Examples further illustrate the present invention but should not he construed as in any way limiting its scope.

### EXAMPLES

### Example 1

### HPLC Quantitation of Gallium

Approximately 0.7 g of gallium (Ga) was weighed into a 50 ml volumetric and made up to volume with deionised water (18.2 mΩ) to make a stock solution equivalent to 4 mg/ml Ga³⁺. Serial dilutions were performed to produce a range of standard solutions between 4 mg/ml and 0.125 mg/ml. The standards were transferred to 2 ml crimp sealed glass HPLC vials prior to injection. A mobile phase (pH 4.2) stock solution was prepared using 7.0 mM pyridine dicarboxylic acid (PDCA), 66 mM potassium hydroxide, 5.6 mM potassium sulphate and 74 mM formic acid. The mobile phase stock solution was diluted 1 in 5 with deionised water (18.2 m′Ω) then filtered and degassed for one hour prior to use. The post-column reagent (pH 10.4) stock solution was prepared using 1.0 M 2-dimethylaminoethanol, 0.5 M ammonium hydroxide and 0.3 M sodium bicarbonate, 4-(2-pyridylazo) resorcinol (PAR) was added prior to use to make the final solution (0.12 g per 1000 ml), but this must be freshly prepared daily and stored under nitrogen to prevent oxidation.

The IONPAC® CS5A column was used with a HP 1050 Liquid Chromatogram (Agilent, Wokingham) set with an injection volume of 50 μL. The mobile phase flow rate was 1.2 ml/min and the column temperature was 60° C. The column eluent was reacted with PAR in a 375 μL post-column reaction coil to allow PAR-drug complexation prior to detection with a UV 975 UV/Vis detector (Jasco, Great Dunmow) at 5.30 nm. The post-column reagent flow rate was controlled under nitrogen at 0.6 ml/min using a PC10 pneumatic controller (Dionex corp., Sunnyvale, US), resulting in a total flow rate of 1.8 ml/min within the reaction coil.

The suitability and efficiency of the system was assessed by calculating peak symmetry and theoretical plates using the following equations:

Peak symmetry

$$As = \frac{{W}_{0.1}}{2 d} ;$$

wherein W_{0.1}=peak width at one-tenth peak height and D=distance between perpendicular dropped from peak max and leading edge of peak at one-tenth peak height.
Theoretical plates

$$n = {5.54 [ \frac{{T}_{r}}{{W}_{h / 2}} ]}^{2} ;$$

wherein Tᵣ=drug retention time and W_{h/2}=peak width at half peak height.

To test the precision of the method various standards were assayed in series. Six injections of each sample was performed (n=6), preceded by a blank of deionised water (18.2 m′Ω). A single set of standards were analysed three times consecutively (intra-day variation). Three freshly prepared sets of standards were injected on three separate days (inter-day variation). From this data the precision of this method was assessed and calibration curves were constructed to determine the linearity, sensitivity, limit of detection (LOD) and limit of quantification (LOQ).

The LOD and LOQ were calculated from the calibration carve using the following equation.

LOD=Y_{B}+3S_{B} Limit of detection

LOD=Y_{B}+10S_{B} Limit of quantification

Y_{B}—Y intercept from regression equation
S_{B}—standard error of the Y estimate

As it was necessary to increase the injection volume to achieve the required sensitivity, a range of gallium samples between 0.125 and 0.007813 mg/ml were injected (n=3) using an injection volume of 100 and 200 μL.

The accuracy of the assay was assessed using equation below by injecting five known concentrations sample into the column and calculating the theoretical concentration from the standard calibration curve.

$$Accuracy = [ \frac{T}{A} ] \times 100$$

T=theoretical sample concentration
A=actual sample concentration

The IONPAC® CS5A column is a mixed anion-cation exchange column with sulfonic acid and alkonol quaternary ammonium functional groups. PDCA masks the positive charge of the gallium ions by forming stable anionic complexes reducing the attraction between the gallium ions and the stationary phase. The column eluent was reacted with PAR in a 375 μL post-column reaction coil, prior to detection. PAR displaced the PDCA from the metal complex and became oxidized. It is the extent of this oxidation that was detected.

A single GN peak was detected with a retention time of 2.57±0.02 min. The peak shape for GN displayed tailing in all chromatograms with an average A_{S} value of 1.72 (n=100). This tailing was consistent with the manufacturer's sample chromatograms for other transition metals. Column efficiency was estimated using theoretical plates, n=514 (n=10) and was used to ensure column performance did not deteriorate over time.

Peak height (R²=0.9931) showed inferior linearity between 2 and 0.125 mg/ml compared to peak area (R²=0.9988) in this method. As a result peak area was deemed to be a more reliable estimate of gallium concentration over this range of concentrations and was used in all further experimentation. However, as the % CV of the Ga standards was 5% and therefore over the acceptable standard for precision which is 2%, calibration curves requires construction daily (Health Canada, 1994),

The LOD for gallium with an injection volume of 50 μL was calculated to be 71.1 μg/ml. Previously reported skin studies involving inorganic ions (DeNuzzio & Berner, 1990) suggested that a sensitivity of ≦6 μg/ml was required, while others have suggested that 10% of the applied dose of small metal ions may penetrate human skin (van Hoogdalem, 1998). Considering Ga is small it was estimated that a sensitivity ≦10 μg/ml would be required, Thus, it was necessary to increase the injection volume from 50 μL to 100 μL, to attempt obtaining these detection limits.

Increasing the injection volume did not affect linearity (0.998) for a range of Ga standards between 0.125 and 0.007813 mg/ml in deionised water but, it did reduce the limit of detection. Injection volumes of both 100 and 200 μL achieved the predicted sensitivity limit of 10 μg/ml discussed above. However, an injection volume of 300 μL had the lowest LOD of 5.37 μg/ml and also the greatest linearity.

Using the peak area for quantification, the mean value for the accuracy of the method within the range of 0.4 and 0.1 mg/ml was 99.58±5.46% as shown in Table 1 below. Table 1 shows the mean theoretical (from calibration) and actual (standard solution) sample concentrations (n=3) showing gallium HPLC assay method accuracy. Ga concentration was calculated from peak area and an injection volume of 100 μL within a range of 0.4 to 0.1 mg/ml.

TABLE 1

| Actual Ga | Theoretical Ga | |
|---|---|---|
| concentration (mg/ml) | concentration (mg/ml) | Accuracy (%) |
| 0.4 | 0.41 | 102.8 ± 1.63 |
| 0.3 | 0.31 | 102.1 ± 3.46 |
| 0.2 | 0.21 | 105.3 ± 6.28 |
| 0.15 | 0.14 | 95.54 ± 3.89 |
| 0.1 | 0.094 | 94.16 ± 4.99 |

### References

Health Canada, Drugs Directorate Guidelines; Acceptable Methods 1994, Health Protection Branch, published by authority of the minister of National Health and Welfare, pp 1-53.

DeNuzzio, J. D, & Berner, B. 1990, “Electrochemical and iontophoretic studies of human skin”, Journal of Controlled Release, 11 (1-3): 105-112.

van Hoogdalem, E. J. 1998, “Transdermal absorption of topical anti-acne agents in man; review of clinical pharmacokinetic data”, Journal of the European Academy of Dermatology and Venereology, 11 (S1); 13-19.

### Example 2

### Chemical Stability of Gallium Nitrate (GN) in Different Buffers

The chemical stability of Ga in different 50 mM buffers was assessed including TRIS (pH 7.0), pyrrolidine (pH 11.3), citric acid (pH 5.0), borate (pH 8.0), formic acid (pH 2.5), phosphate (pH 7.3) and HEPES (pH 7.2). Three standard solutions of Ga in each buffer was assessed by HPLC analysis using the method described in Example 1 at four time points 0, 24, 48 and 100 h. Performance was measured by percentage gallium recovery according to the following formula:

$$percent recovery ( \% ) = \frac{sample conc .}{standard conc .} \times 100$$

To further test chemical stability, gallium recovery from the citric acid buffer (pH 5.0, 50 mM) containing pig cheek skin samples (0.6 g±0.025) was assessed. The skin samples were placed in three concentrations of GN in citric acid buffer (n=3). The samples were stirred constantly at 37° C. and 1 ml samples were withdrawn and assayed after 0, 24, 48 and 100 h to assess gallium recovery.

As shown in Table 2 below, pyrrolidine, citric acid and formic acid displayed Ga recovery similar to the water control. The other systems investigated TRIS, borate, phosphate and HEPES, were incompatible with Ga. The TRIS, HEPES and phosphate buffers turned cloudy on mixture and the Ga recovery being below that of water. The low recovery of Ga was probably due to the well documented formation of insoluble, amorphous gallium phosphate (Chang & Pearson, 1964) and the tendency of gallium to bind with proteins via intermolecular interactions (Rudnev et al., 2006). The borate buffered solution did not turn cloudy however, no peak was observed with the HPLC method. This may be explained by the formation of very stable polyborate gallium complexes (Ding et al., 2004).

TABLE 2

| Buffer (50 mM) | [Ga] mg/ml recovered | | |
|---|---|---|---|
| Water (pH 7.0) | 0.5 | 0.25 | 0.13 |
| control/target conc. | | | |
| TRIS (pH 7.0) | 0.49 | 0.13 | 0.01 |
| Pyrrolidine (pH 11.3) | 0.49 | 0.25 | 0.13 |
| Citric acid (pH 5.0) | 0.54 | 0.28 | 0.15 |
| Borate (pH 8.0) | no peaks observed | | |
| Formic acid (pH 2.5) | 0.55 | 0.29 | 0.16 |
| Phosphate (pH 7.3) | 0.30 | 0.11 | 0.04 |
| HEPES (pH 7.2) | 0.13 | 0.06 | 0.10 |

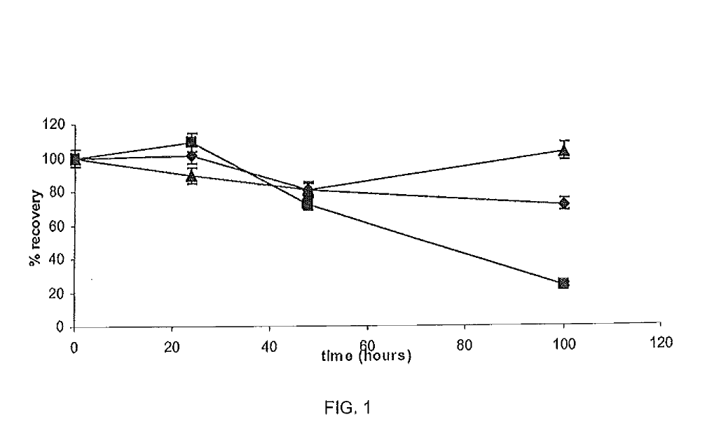

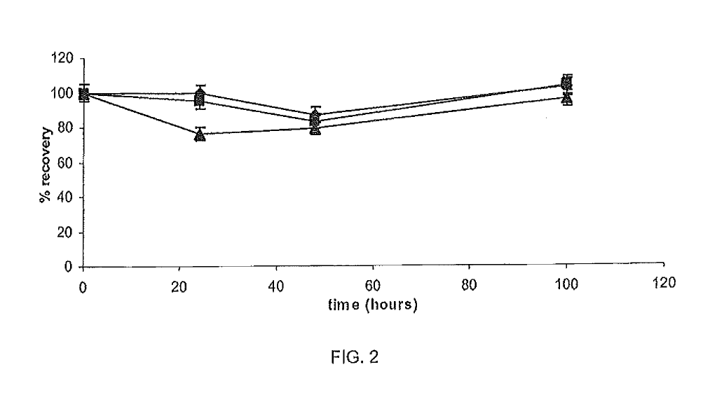

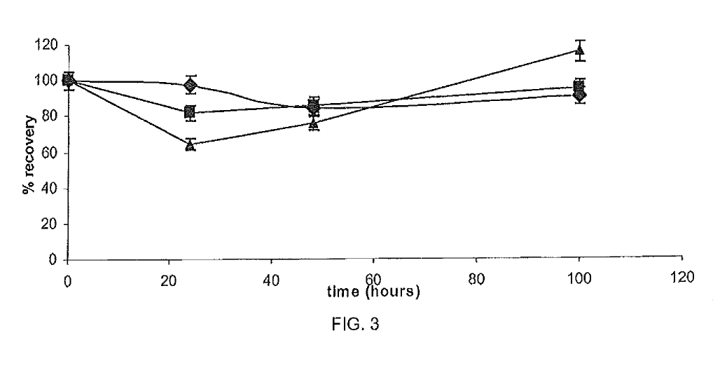

As shown in FIGS. 1, 2 and 3, respectively, the pyrrolidine buffer had a mean % recovery of 66.1±60.2%, formic acid 99.89±13.5% and citric acid buffer was 100.6±4.1%. No significant Ga loss was observed between 0 and 100 h for Ga dissolved in citric or formic acid buffer (P>0.05, ANOVA), however a significant Ga loss was observed with 0.25 mg/ml Ga dissolved in pyrrolidine buffer (P<0.05, ANOVA) after 100 h.

### References

Chang, L. L & Pearson, G. L. 1964, “The solubilities and distribution coefficients of Zn in GaAs and GaP”, Journal of Physics and Chemistry of Solids, 25(1): 23-30.

Rudnev, A. V., Foteeva, L. S., Kowol, C., Berger, R., Jakupee, M. A., Arion, V. B., Timerbaev, A. R., Keppler, B. K. 2006, “Preclinical characterization of anticancer gallium (III) complexes: solubility, stability, lipophilicity and binding to serum proteins”, Journal of Inorganic Biochemistry, 100(11): 1819-26.

Ding, X. X., Huang, Z. X., Huang, X. T., Gan, Z. W., Cheng, C., Tang, C., Qi, S. R. 2004, “Synthesis of gallium borate nanowires”, Journal of Crystal Growth, 263(1-4): 504-9.

### Example 3

### Iontophoretic Delivery of Gallium Nitrate into the Epidermal Sheet of Human Skin

Human skin was obtained with patient's informed consent from abdominoplasties and frozen at −30° C. Human epidermal sheet was prepared from the full thickness skin by placing the full thickness skin in a glass beaker of deionised water (DiH₂O) (0.5-1.0 μS/cm) at 60° C.±3° C. for 60 seconds. The skin was then placed dermal side down on aluminium foil to allow the epidermal layer to be gently rolled back with the thumb. The removed epidermal sheet was floated in a pan of DiH₂O (stratum corneum facing up) allowing a sheet of filter paper to be eased underneath (Kligman A. M. and Christophers E., 1963). The filter paper was removed with the epidermal sheet adhered, smoothing any kinks in the skin. The mounted epidermal sheet was finally wrapped in aluminium foil and frozen at −30° C. until required. The permeation of gallium across epidermal human skin was investigated using previously calibrated upright small Franz cells of approximately two mL volume (area of 0.6 cm², MedPharm Ltd., Guildford, UK) both passively and after iontophoresis. Iontophoresis was applied to the appropriate cells with a current of 400 μA for 10 min using the Labion Model MI-200 Iontophoretic Medicator attached to copper wire anodes and cathodes. Citric acid buffer (pH 5.0, 50 mM) was used as the receiver fluid in the Franz cells with a magnetic stirrer. A saturated gallium solution (16.7%) was applied to the donor compartment of the Franz cell (n=8-12). Sample volumes of 0.5 mL were removed at the appropriate time points and replaced with thermostatically regulated receiver fluid. The studies were performed over 24 h with intermittent sampling points. The samples were assayed using the LC ion-exchange method.

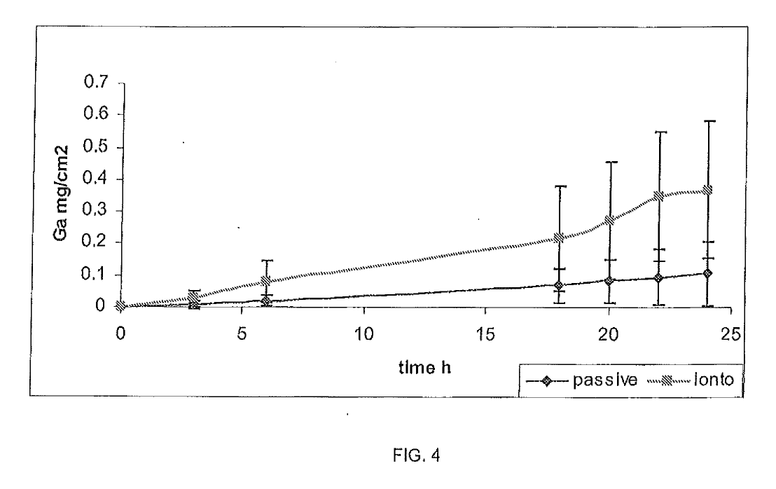

After 24 hours of passive permeation using a saturated 16.7% w/v gallium nitrate solution (FIG. 4), the mean gallium permeation across human epidermal sheet was 0.11±0.09 mg/cm². The cells which received 10 min of iontophoresis displayed a mean permeation of 0.37±0.21 mg/cm² after the same 24 hour period. The application of the iontophoretic charge caused a significant increase (P<0.05, ANOVA) of 240% in gallium flux across the human epidermal sheet over 24 hours.

### References

Kligman A. M., Christophers E., 1963. Preparation of isolated sheets of human stratum corneum. Arch Dermatol, 832-833.

### Example 4

### Iontophoretic Delivery of Gallium Nitrate into Full Thickness Human Skin

Human full thickness skin was stored in the same fashion as the epidermal sheet (Example 3), and a similar Franz cell assembly technique was adopted. The effect of concentration was investigated by applying 16.7, 1.5 and 0.15 % w/v gallium solutions in pH 5.0 citric acid buffer to the donor compartment (0.8 mL). Ten minutes of 400 μA iontophoresis was applied to all of the cells and 0.5 mL samples were removed after 0, 24, 28, 42, 65 and 70 h and replaced with receiver fluid. The gallium in the samples was assayed using the LC ion-exchange method. Separately, saturated gallium solutions (16.7%) were applied to donor compartments and the cells were then either subjected to 0 (passive), 10 min or 60 min of 400 μA iontophoresis (n=8). Samples of 0.5 mL were removed for LC ion-exchange assay after 0, 24, 28, 42, 52 and 70 h and replaced with receiver fluid. The diffusion conditions are summarized in Table 3. The binding affinity of gallium for full thickness human skin was calculated by measuring the gallium recovery from skin pieces of known mass. Gallium solutions containing 2.4, 1.6, 0.79, 0.395 and 0.1975 mg of gallium in 20 μL of water were applied to the stratum corneum of the skin samples using a positive displacement pipette (Eppendorf, Cambridge, UK). The samples were then left for 24 h to allow the gallium to permeate the skin. The skin samples were then finely sliced using scissors, chilled and homogenised for five minutes using a T10 Ultra-Turax homogeniser (IKA works, Staufen, Germany) using 30 second pulses. The scalpel, scissors, forceps and homogeniser blades were washed through using 10 mL of PDCA mobile phase concentrate as the extractant. The PDCA concentrate (pH 4.2) was freshly made every week by weighing approximately 5.8 g PDCA, 18.5 g potassium hydroxide, 4.9 g potassium sulphate and 17.0 g of formic acid into a 1000 mL volumetric and making up to volume with DiH₂O. The skin-PDCA concentrate homogenate was allowed to mix for a further 24 h before being filtered using 0.45 μm, 30 mm cellulose acetate syringe filters (Orange Scientific, Braine l'Alleud, Belgium). The gallium in the remaining solution was assayed using the LC ion-exchange method. The binding affinity was calculated by subtracting the amount of gallium recovered by the extraction from the amount applied to the skin, in order to gain the amount of gallium still bound to skin in the homogenate. This figure was then divided by the mass of the skin sample to calculate the amount of gallium bound per gram of skin.

An infinite dose mass balance study was performed using full thickness human skin. Small Franz cells were set up as previously described and the mass and thickness of skin section recorded. A 1.5 % w/v gallium solution was applied (0.6 mL) to the donor compartment of each cell. Cells were then treated with either 60 mm iontophoresis or received no iontophoresis (n=6 for each). The gallium solution was left in contact with the skin for the required duration and the cells were then carefully dismantled. The donor and receiver cells were sampled for gallium assay by LC ion-exchange. The cells were then swabbed with PDCA mobile phase concentrate soaked cotton buds to remove any remaining gallium. The skin samples were removed and reweighed. The upper surface of the skin was stripped with Scotch tape 19×50 mm (3M, Bracknell, UK) 3 times to remove surface adhered donor solution. The remaining skin samples were homogenised as previously described to determine the gallium level in the skin. The gallium recovery from the skin was corrected to account for gallium binding.

TABLE 3

| Parameter | Condition |
|---|---|
| Skin sample | Full thickness human skin; defatted |
| | abdominoplasty |
| Franz cell | Small. Volume is approximately 2 ml. |
| | Area is approximately 0.6 cm² |
| Donor solution | 16.67% (saturated), 1.5%, and 0.15% |
| | gallium (III) nitrate in pH 5 citric acid |
| | solution (0.8 ml) |
| Receiver solution | pH 5 citric acid solution at 37° C. with |
| | constant stirring |
| Iontophoretic conditions | 400 uA for 0, 10 or 60 minutes; |
| | Current density is approximately 650 uA/cm² |
| Sampling | 0.5 ml, replaced with calibrated receiver |
| | fluid for duration of 70 h |
| Detection | Gallium HPLC assay |

### Effect of Concentration

Gallium was only detected in the receiver, fluid above the LOD after the 24 hour time point, thus the duration of the experiment was increased to 70 h. After 70 hours of thermostatically controlled passive permeation using a saturated gallium nitrate solution (Table 4) the mean amount of gallium to permeate full thickness human skin was 0.004±0.011 mg/cm². No gallium was detected in the receiver fluid in seven out of the eight cells. Even after 70 h only the saturated gallium solution delivered enough gallium through the skin to allow detection and only in one cell. As a result of this the effect of gallium concentration was just investigated with the application of iontophoresis.

TABLE 4

| Gallium | | | | Amount of Ga |
|---|---|---|---|---|
| concentra- | Degree of | Duration of | | detected in |
| tion | saturation | iontophoresis | Steady state Ga | receiver after 70 h |
| (% w/v) | (%) | (min) | flux (μg/cm²/h) | (mg/cm²) |
| 16.67 | 100 | 0 | 0.009 ± 0.003 | 0.004 ± 0.011 |
| 16.67 | 100 | 60 | 1.300 ± 0.450 | 0.063 ± 0.022 |
| 16.67 | 100 | 10 | 0.900 ± 0.238 | 0.049 ± 0.013 |
| 1.5 | 9 | 10 | 0.900 ± 0.695 | 0.044 ± 0.034 |
| 0.15 | 0.9 | 10 | 0.008 ± 0.005 | 0.0028 ± 0.006 |

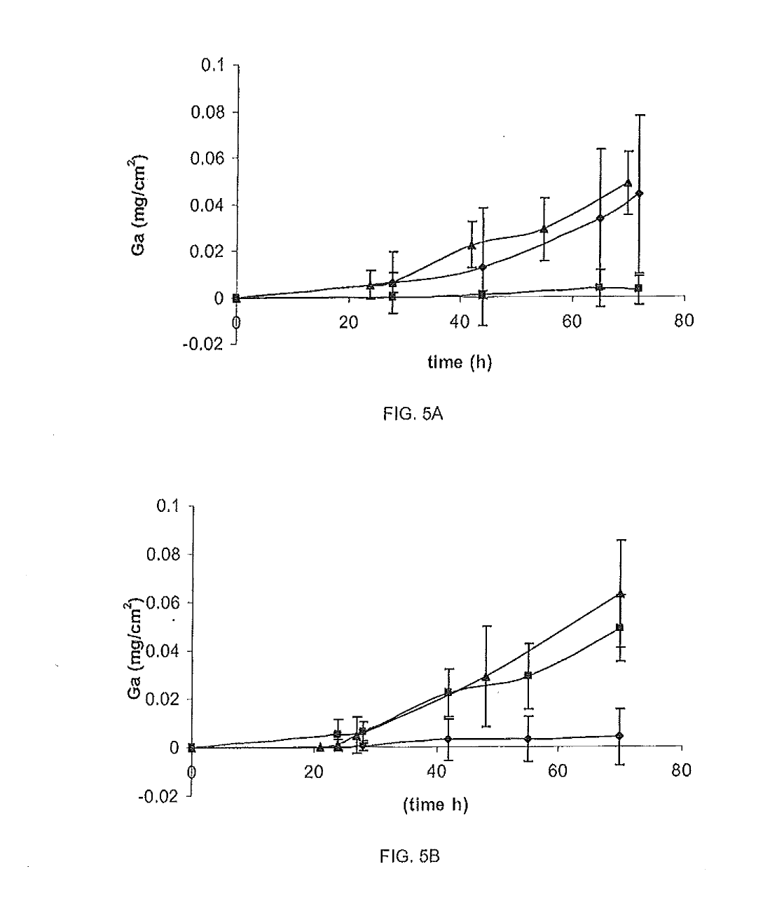

The application of 10 min iontophoresis to a saturated gallium nitrate solution (16.67% w/v) resulted in 0.049±0.013 mg/cm² of gallium being detected in the receiver fluid after 70 h (Table 4, FIG. 5A). In comparison 0.0438±0.034 mg/cm² was detected for a 1.5% w/v gallium solution and 0.0028±0.006 mg/cm² for a 0.15% w/v solution. A 10 fold decrease in the gallium concentration between 16.67% w/v and 1.5% w/v Ga only reduced the amount of drug permeating the skin by 10.6%; this decrease was not statistically significant (P>0.05, ANOVA). The steady state flux was determined from the slope of the curves over five time points. The steady state flux for both 16.67% w/v Ga and 1.5% w/v Ga was found to be 0.9 μg/cm²/h, this reduced to 0.008 μg/cm²/h for 0.15% w/v Ga formulations.

### Effect of Iontophoresis Duration

The application of 10 min of iontophoresis to the saturated gallium solution (16.7% w/v) increased the mean gallium permeation to 0.049±0.013 mg/cm² after the same 70 hour period, a 1125% increase compared to the passive cells. The application of 60 min iontophoresis to the saturated gallium solution resulted in an average of 0.063±0.022 mg/cm² of gallium being detected in the receiver fluid, an increase in gallium permeation of 1475% within 70 hours compared to the passive cells. The application of 10 and 60 min iontophoresis therefore significantly increased gallium permeation (P<0.05, ANOVA) across human skin when compared to passive permeation. The steady state flux across the skin was determined over five time points for each set of iontophoretic conditions. Passive permeation had a steady state gallium flux of 0.009±0.003 μg/cm²/h. This increased to 0.9±0.238 μg/cm²/h after the application of 10 min iontophoresis. The steady state flux of 1.3±0.450 μg/cm²/h was observed in the cells which received 60 min of iontophoresis (Table 4, FIG. 5B).

### Binding Affinity

A known amount of gallium in citric acid buffer (pH 5.0) was delivered using a positive displacement pipette to the apical surface of the skin and allowed to diffuse into the skin for 24 h. The skin was then homogenised and the gallium recovery determined by extraction with PDCA mobile phase concentrate prior to HPLC assay. Determination of the specific binding affinity of human full thickness skin for gallium is outlined in Table 5 and was determined to be 0.17±0.03 mg/g.

TABLE 5

| | | | | Ga | Skin | |
|---|---|---|---|---|---|---|
| | Ga | Ga | | bound | sample | |
| | delivered | recovered | % Ga | to skin | mass | Binding |
| Sample | (mg) | (mg) | recovery | (mg) | (g) | (mg/g) |
| A | 2.4 | 2.33 | 97.1 | 0.07 | 0.506 | 0.138 |
| B | 1.6 | 1.48 | 92.5 | 0.12 | 0.674 | 0.178 |
| C | 0.79 | 0.69 | 87.3 | 0.1 | 0.517 | 0.193 |
| D | 0.395 | 0.29 | 73.4 | 0.105 | 0.534 | 0.197 |
| E | 0.1975 | 0.12 | 60.8 | 0.0775 | 0.513 | 0.151 |
| Mean | | | | | | 0.171 |
| s.d. | | | | | | 0.026 |
| % CV | | | | | | 15.1 |

### Human Skin Recovery

Mass of gallium detected in the skin after passive and iontophoretic (400 μA for 60 min) delivery as a measure of gallium diffusion into the skin is described in Table 6. (Cells dismantled after 0 and 60 h post iontophoresis). After 60 hours, a three fold increase in skin gallium content is observed when comparing iontophoretic and passive gallium skin permeation (0.617±0.059 and 0.221±0.176 mg/cm² respectively) using a 1.5% w/v gallium solution. Immediately after the application of iontophoresis the detected gallium level in the skin was only increased by 12% (iontophoresis=0.200±0.069 and passive=0.178±0.058 mg/cm²). After a duration of 60 h, gallium levels detected within the full thickness skin samples and gallium permeation through the skin were both increased by a proportional amount after the application of iontophoresis. An approximate three-fold increase in the gallium flux was observed with 60 min iontophoresis, both in the Franz cell receiver compartment and after recovery from the skin, compared to passive. The total amount of gallium detected in the skin was ten-fold higher than the levels detected in the receiver compartment of the Franz cells in both passive and iontophoretic cells.

TABLE 6

| | Ga mass detected | | |
|---|---|---|---|
| | | Skin | Per |
| | Full thickness | corrected for | skin surface |
| Conditions applied | human skin (mg) | binding (mg) | area (mg/cm²) |
| Ionto 60 h (n = 6) | 0.328 ± 0.371 | 0.391 ± 0.373 | 0.617 ± 0.059 |
| Passive 60 h (n = 6) | 0.0745 ± 0.108 | 0.137 ± 0.109 | 0.221 ± 0.176 |
| Ionto 0 h (n = 5) | 0.0788 ± 0.043 | 0.127 ± 0.043 | 0.200 ± 0.069 |
| Passive 0 h (n = 5) | 0.07 ± 0.036 | 0.111 ± 0.035 | 0.178 ± 0.058 |

### Example 5

### Effect of Gallium Coordination on Gallium Permeation Across Human Skin

### Abstract

Gallium nitrate has been shown to up-regulate the production of collagen which may have potential benefits for wound healing and reducing the effects of photodamage and aging. Although it has previously proven impossible to model the topical application of metals, little emphasis has been placed on the role of coordination complexation in percutaneous permeation. Using Hyperquad Simulation and Speciation software (HYSS) speciation plots to predict the species of gallium present, it was demonstrated that the metal complex which gallium forms affects the rate at which gallium permeates full thickness human skin. It was shown that free gallium ions passively permeate the skin four times faster than gallium incorporated in citrate or hydroxide complexes; however permeation of free ions is notoriously difficult to control. Furthermore, it was demonstrated that the application of anodal iontophoresis can increase the flux of gallium across the skin by up to 80.000% compared to passive permeation, when applied to a mixture of positively charged gallium hydroxide complexes. In addition, the gallium permeation of negatively charged gallium citrate complexes across the skin can be increased by 8.200% compared to passive permeation by the addition of cathodal iontophoresis. Full thickness skin studies have shown that elemental gallium skin deposition is increased from 0.288±0.258 mg/cm² with passive delivery to 1.203±0.248 mg/cm² with 60 min of anodal iontophoresis. This further increased to 1.888±1.159 mg/cm² after 24 h of passive permeation following iontophoresis. It was also shown that elemental gallium skin deposition is 1.281±0.27 mg/cm² after 60 min of cathodal iontophoresis of the gallium citrate donor solution, which increased to 1.550±0.352 mg/cm² after 24 h of passive permeation following iontophoresis. The gallium deposition levels achieved from iontophoretic delivery of targeted gallium species were in excess of maximum desired concentration ranges published (0.25-100 uM) (Bockman et al. 1993; Goncalves et al. 2002). These results suggest that gallium co-ordination complexes can be optimized for optimum iontophoretic delivery into and through the skin.

### A. Introduction

Investigating the penetration of compounds across intact skin is of great interest to both pharmaceutical and cosmetic scientists. Although the stratum corneum (SC) is only 10 μm thick, it is this continuous layer that forms the major barrier to drug permeation. The SC is formed from rigid, flattened, cornified ‘dead’ keratinocytes stacked to form dense overlapping multiple layers of cells held together by desmosomes. The tightly packed, interlinked cells provide a highly permselective barrier that does not contain any active transport mechanisms. Compounds pass through if via passive transport and therefore this process can be modelled, considering several assumptions, using Higuchi's Law (Equation 1).

$$\frac{δ q}{δ t} = \frac{α}{γ} \frac{DA}{L} \qquad Equation [ 1 ]$$

Where the steady state rate of drug penetration (δq/δt), is related to the thermodynamic activity of the drug in the vehicle (α), the activity coefficient of the drug in the skin barrier phase (γ), the effective diffusivity in the barrier phase (D), the effective thickness of the skin (L) and the skin surface area being treated (A) (Higuchi 1959). The greatest rate of passive penetration is obtained by using saturated or supersaturated solutions from which the drug is readily available and the thermodynamic potential is relatively high. The degree of saturation is more important than absolute concentration alone, as complex compounds may form crystalline structures of differing free energy, whereby the most energetic species will penetrate the skin at a faster rate. It follows that vehicles with lower affinity for the drug will produce a faster rate of penetration.

Iontophoresis (ITP) uses the application of an electrical current to enhance the delivery of drugs to the skin. It is especially effective when applied to small, lipophilic, positively charged drugs (Barry, 2002). It is based on the electrical theory that ‘like’ repels ‘like’ (Singh & Maibach, 1996), hence the application of a positive charge will drive positively charged drug molecules through a barrier. There are two other proposed modes of action for enhancing dermal delivery using iontophoresis: 1) electroosmosis, the transportation of polar neutral molecules by water convection and 2) electropertubation, which causes an alteration in the orientation of the lipid molecules, and as a result increasing the skin's permeability to both charged and uncharged species (Barry, 2001). This alteration in the skin induced by electropertubation is reportedly reversible, but can last for some time after the removal of the electrical stimulation. The transport of a drug across the skin using ITP can involve any combination of these three mechanisms.

The permeability of a wide range of metals into and through the skin has previously been studied due to their importance in toxicology and immunology (Fullerton & Hoelgaard, 1988). It has been shown that most transition metals are able to penetrate the SC to some degree; however, the rate and extent is unpredictable and difficult to control, with steady state conditions as defined by Higuchi's theory rarely observed. As a result, it has not been possible to define a generic model that predicts the permeation of metals through the skin, nor has it been viable to define their quantitative structure-diffusion relationships (Hostynek 2003). In addition, there has traditionally been little harmony between the methods of investigation employed in this field, with few published articles including enough data for a permeability coefficient (K_{P}) to be calculated (Hostynek et al., 1993).

Ga administered as gallium nitrate has been shown to up-regulate collagen and fibronectin expression in human dermal fibroblasts in vitro (Bockman et al., 1993) and promote re-epithelialization in a porcine partial thickness wound model in vivo (Goncalves et al., 2002). The primary effect of Ga on collagen up-regulation is the promotion of fibroblast migration by increased gene expression of the early wound structural proteins fibronectin and type I procollagen (Briggs, 2005) It is also thought that Ga may inhibit extra-cellular matrix (ECM) metalloproteinase (MMP) activity by displacing zinc which is required as a cofactor. MMPs are enzymes which degrade proteins in the ECM controlling homeostasis hence, blocking these enzymes may result in a net increased ECM accumulation (Goncalves et al., 2002), It has been postulated that Ga may exert a similar effect on intact skin to that in wounds (Goncalves et al., 2002). Increasing type I procollagen expression in intact skin may produce a variety of desirable outcomes such as increased skin thickness, tensile strength, and elasticity. The potential benefits of delivering gallium to intact skin may include reduction in photodamage, where the collagen and elastin function is impaired by over exposure to UV light from the sun. In addition, increased skin thickness may be of benefit to the elderly and patients with adrenal or pituitary disorders such as Cushing's syndrome, a condition characterised by skin thinning and weakness. Skin atrophy is also a documented side effect of long term use of corticosteroids (e.g. methylprednisolone). In addition, alleviation of wrinkles for cosmetic purposes may be of benefit. While the effect of Ga is yet to be substantiated in unwounded skin models, its effects in wounded skin models, in which gene expression and up-regulation cascades are already initiated by complex interactions involving many enzymes and biological factors, have already been demonstrated. It remains to be seen whether Ga deposition in the skin can initiate an increase in collagen synthesis by fibroblasts in intact skin.

No previous evidence of gallium permeation into human skin has been reported. Animal studies using topical application have been performed, however there was no attempt at measuring the rate of Ga permeation, with skin deposition assumed to be equivalent to applied dose (Goncalves et al., 2002). This was also the case for the most closely related chemical entity, iron. Although iron is essential for DNA and RNA synthesis and has been used in dermatology for acne and alopecia, reliable percutaneous absorption has only been reported for the chelated Fe-cupferron form whereby only 10-15% was absorbed (Hostynek et al., 1993). It was also noted that ionic iron has a great affinity for nucleophiles and becomes highly protein bound. For these reasons it was believed that delivering adequate quantifiable levels of the small, charged Ga molecule into the dermis would be problematic and that the use of a physical permeation enhancer such as ITP would be of benefit.

Ga is present as a trivalent ion in simple aqueous solution, but being a hard acid has a tendency to form chelates through bonds with oxygen and other ligands that are present such as citrate. In order to gain the most efficient gallium skin permeation, it was predicted that the species of gallium delivered must be controlled. To minimise the effects of charge and size exclusion and maximise the effect of ITP delivery the investigation of various gallium donor species was necessary. Optimising topical gallium delivery requires knowledge of all the gallium species present in the formulation. It was possible, by entering the stability constants of all the possible chemical entities present in a system into a computer model, to predict the species that would be formed at specific conditions (Alderighi et al., 1999). The species of gallium formed within a given system is a function of pH, competing ion concentration and the relevant equilibrium constants. Therefore, by controlling the pH and concentration, it is possible to predict which species will predominate within any given simple system thus enabling the optimisation of the Ga donor species for delivery into the skin.

The aim of this study was to investigate the effect of gallium speciation on the extent of gallium permeation through and deposition into the skin both passively and after the application of ITP. Different gallium donor solutions were examined in order to maximise and control gallium delivery and deposition at the target site, with the goal of achieving a suitable concentration so as to up-regulate collagen synthesis.

### B. Materials

Gallium (III) nitrate hydrate (99.9%), pyridine dicarboxylic acid (PDCA), potassium hydroxide, potassium sulphate, formic acid, 2-dimethylaminoethanol, ammonium hydroxide solution, sodium bicarbonate, 4-(2-pyridylazo) resorcinol (PAR) and citric acid were all obtained from Sigma Aldrich (Gillingham, UK) and were of reagent grade. Scintillation fluid (Hionic Fluor) was supplied by Perkin Elmer (Bucks, UK), while Tritiated water (³H₂O) was purchased from Amersham Biosciences (Bucks, UK). The IONPAC® CS5A ion-exchange 4×250 mm analytical column and IONPAC® CG5A guard column were purchased from the Dionex corp. (Sunnyvale, US). Reagents were filtered with a Sartorius filter unit (Goettingen, Germany) prior to being degassed. Volumetric flasks and clear glass high performance liquid chromatography (HPLC) vials (2 mL) with lids were obtained from Fisher (Loughborough, UK).

### C. Methods

### (i) Effect of pH on Skin Permeability

The effect of pH on human skin permeability was investigated using previously calibrated upright Franz cells (approx. 0.65 cm² active diffusion area) with a receiver compartment volume of approximately 2 mL (MedPharm Ltd, Guildford, UK). Human skin was obtained with patient's informed consent from abdominoplasty surgery and frozen at −30° C. Sections of approximately 1 cm in diameter were prepared by blunt dissection. Excess fat was removed and the skin was allowed to completely defrost. The skin was attached to the lip of the Franz cell receiver compartment, with the SC facing the donor compartment. The skin was anchored using a glass flange top and bottom. Franz cells were held together with four pieces of parafilm wrapped tightly around the flange top and bottom and then clamped together with a Quickfit clip. The Franz cells were placed in a water bath at 37° C. to ensure a skin surface temperature of 32° C. (Maddock & Coller, 1933). Citric acid buffer at either pH 2 or pH 5 (0.5 M) was used as the receiver fluid in the Franz cells with a magnetic stirrer (n=4). The assembled cells were allowed to equilibrate for one hour prior to donor fluid application. A donor solution of tritiated water in buffer solutions pH 2.0 or pH 5.0 (n=4) (3.7 Bq/mL) was prepared and 200 μL applied to the donor compartment using a Gilson automated pipette. The donor and receiver compartments were covered using parafilm to reduce radioactivity loss due to evaporation. Receiver fluid samples of 0.5 mL were withdrawn from the receiver compartment at regular intervals and replaced with thermostatically regulated receiver fluid. The samples were aliquoted directly into scintillation vials to which 4 mL of scintillation fluid was added. The vials were tightly closed, mixed thoroughly by shaking and analysed using a LS 6500 Multipurpose scintillation counter (Beckman Coulter™, Bucks, UK). Radioactivity detected in the receiver compartment was used to determine the rate of flux across the skin and quantified as disintegrations per minute (DPM). The studies were performed for 70 h with samples taken at 0, 3, 6, 22, 28, 33, 48, 54 and 70 h,

### (ii) Gallium Speciation

Hyperquad simulation and speciation (HYSS) software was used to determine the gallium species present in a variety of citrate buffer systems in order to select appropriate donor solution conditions. The stability constants used to construct the HYSS speciation plots were obtained from previous studies for 0.010 M Ga in citric acid at 25° C. (Harris & Martell 1976; Nazarenko et al., 1968). These speciation plots were used to predict the effect of pH and concentration on the gallium species present in different systems. From these plots it was possible to select the vehicle conditions required to gain the desired gallium species for investigating optimum gallium delivery using both anodal and cathodal ITP.

(iii) Effect of Speciation on Gallium Permeation Across the Skin

Solutions of 16.7 and 0.15% w/v gallium in pH 2, 4 and 5 citric acid buffer (1 mM, 50 mM and 1 M) were prepared based on the elemental gallium mass, to generate the gallium species required for delivery, as predicted using the speciation plots. As the application of ITP can affect pH, the pH of the applied gallium systems was measured directly before and after ITP and again after 65 h using a Whatman pH checker (Loughborough, UK). The true pH of the permeating system as measured after application of ITP is reported in the data, as these were the conditions to which the skin was exposed. The effect of gallium speciation on full thickness skin permeation was then investigated using the Franz diffusion cell technique described previously, coupled with the LC-ion exchange assay. The Franz cell receiver compartments (approx. 2 mL) were filled with the equivalent citric acid buffer to be used for the donor solution and allowed to calibrate for 30 min in a thermostatically controlled water bath (37° C.) as before. Infinite doses of 0.8 mL were then applied to the Franz cell donor compartments prior to the application of 60 min of approximately 615 μA/cm² ITP (either anodal or cathodal) to the appropriate cells (n=5). Samples of 0.5 mL were removed after 0, 18, 24, 42 and 65 h and replaced with thermostatically regulated receiver fluid. The gallium in the samples was assayed rising the LC ion-exchange method.

(iv) Effect of Speciation on Gallium Deposition within the Skin

The two gallium donor systems most significantly enhanced by ITP application in the skin permeation studies, one using anodal ITP and one using cathodal ITP, were selected to investigate gallium skin deposition. An infinite dose skin deposition study was performed using full thickness human skin. Small Franz cells were constructed as previously described and the mass of skin section recorded. The Franz cells were set up with a receiver fluid of 0.05 M citric acid buffer (pH 5.0) and allowed to equilibrate for one hour. Donor solutions were prepared and 0.5 mL of each added to the appropriate Franz cell donor compartment. Each cell was subjected to either 60 min of ITP or an equivalent period of passive permeation. The cells were sacrificed after cither 60 min (directly after ITP, n=8) or after 25 h (24 h of permeation, n=8). The gallium solution was left in contact with the skin for the required duration and the cells were then carefully dismantled. Upon sacrifice, the receiver compartment was sampled and gallium concentration determined by LC ion-exchange and checked for leaks. The cells were then swabbed with PDCA mobile phase (5 times concentrate) soaked cotton buds to remove any remaining gallium. The skin samples were removed and reweighed. The upper surface of the skin was stripped with Scotch tape 19×50 mm (3M, Bracknell, UK) 3 times to remove surface contamination. The skin samples were then finely sliced using scissors, chilled and homogenised for five minutes using a T10 Ultra-Turax homogeniser (IKA werks, Staufen, Germany) using 30 second pulses. The scalpel blade, scissors, forceps and homogeniser blades were washed into the homogenate using PDCA mobile phase (five times concentrate) to minimise drug loss from the method. The skin-PDCA concentrate homogenate was allowed to mix for a further 24 h before being filtered using 0.45 μm, 30 mm cellulose acetate syringe filters (Orange Scientific, Braine l'Alleud, Belgium). The gallium in the remaining solution was assayed using the LC ion-exchange method to determine the gallium level in the skill.

### D. Results

### (i) Effect of pH on Skin Permeability

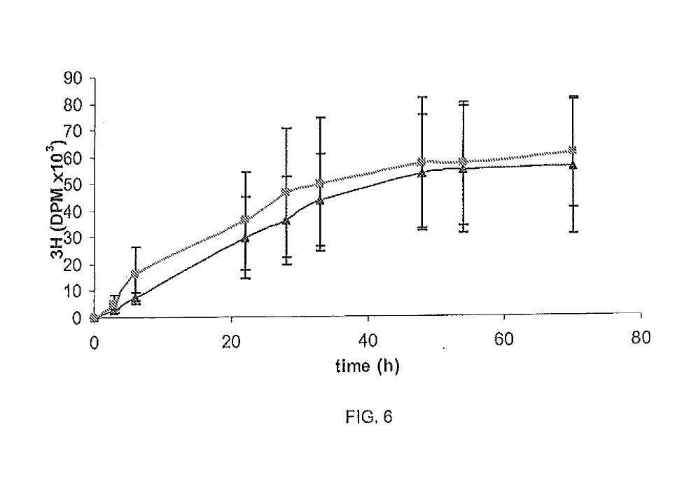

After 70 h of passive permeation at pH 2, an average radioactive count of 55860±25106 DPM/mL (n=4) was detected in the receiver compartment (FIG. 6). This compares to an average count of 60557±20376 DPM/mL (n=4) using tritiated water at pH 5. There was no significant difference (p>0 0.05, ANOVA) in the permeability of the water through human skin between pH 2 and 5 over a period of 70 hours. This enabled the manipulation of the system pH within this range to obtain the desired gallium species without influencing the barrier properties of the skin.

### (ii) Gallium Speciation

Gallium nitrate forms a number of coordination complexes in citrate buffered solutions according to HYSS modelling. It is stable below pH 2 as free cations, but readily forms complexes with citrate and hydroxide ligands forming either charged or neutral complexes at higher pH (as described later). The stability constants for Ga were obtained from previous studies (Table 7). Complex stability increased with stability constant magnitude, whether positive or negative (i.e. further from zero). The complex, stability increased as more hydroxyl groups became coordinated (Table 7). Gallium is able to form the stable negatively charged Ga(OH)₄⁻ species as the molecular spatial arrangement changes from being octahedral to tetrahedral.

TABLE 7
Stability constants for gallium used to construct speciation plots
for gallium in citrate buffer (Benezeth et al. 1997; Nazarenko,
Antonovich, & Nevskaya 1968)

| Gallium species | Log β, 25° C., 0.01M, |
|---|---|
| Ga citrate | 10.02 |
| GaOH⁺⁺ | −2.9 |
| Ga(OH)₂⁺ | −6.6 |
| Ga(OH)₃ | −11.0 |
| Ga(OH)₄⁻ | −15.66 |

### a) Effect of pH

Below pH 2, Ga was almost exclusively present in free ion form, above pH 6 it was mostly present as the negatively charged Ga(OH)₄⁻ species. Between pH 2 and 6 the species present was dependent upon the gallium concentration and buffer strength of the system.

### b) Effect of Ga Concentration

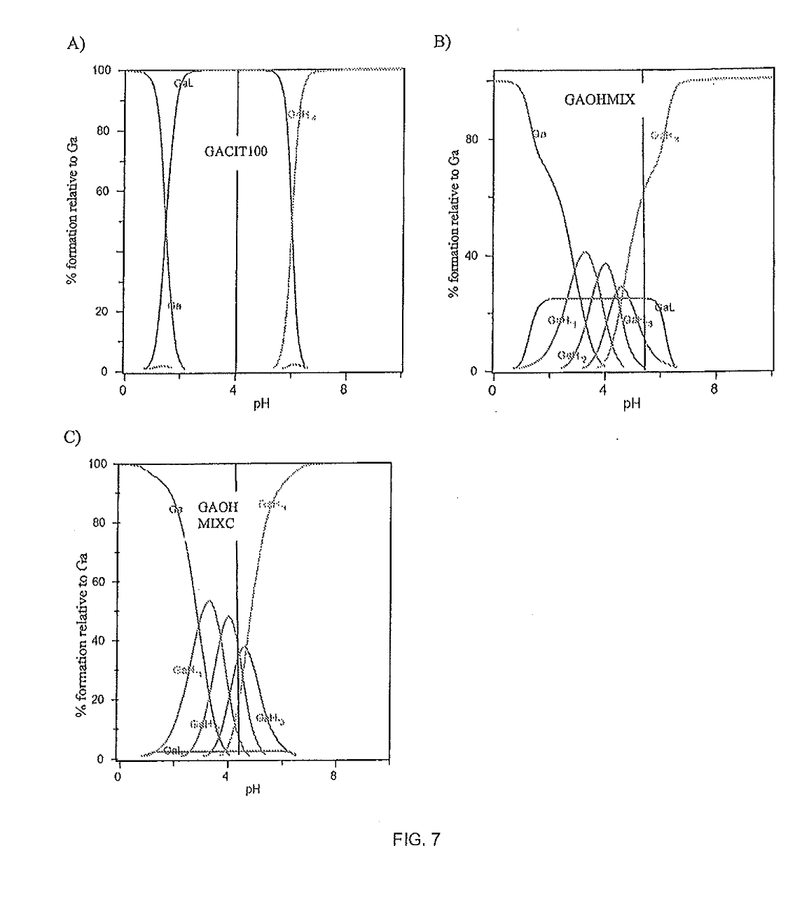

Between pH 2 and 6, using 0.15% w/v gallium in a 0.05 M citrate buffer system gave 100% gallium citrate species as shown in FIG. 7A. However, increasing the gallium concentration 10-fold in the same buffer increased the proportion of gallium ions and hydroxide species present within the pH 2-6 range (FIG. 7B). A concentration of 1.5% w/v gallium in 0.05 M citrate generated a mixture of Ga³⁺ ions and Ga(OH)²⁺, Ga(OH)₂¹⁺, Ga(OH)₃ and Ga(OH)₄⁻ complexes. The 1.5% w/v Ga system gave a larger proportion of the less ionised hydroxide species as the pH increased, but the gallium citrate species remained constant at 25%. As shown in FIG. 7C, Increasing the gallium concentration further to 16.67% w/v (saturation) followed an identical trend, the citrate complex formation was reduced to <5% and the formation of the other species increased proportionally.

### c) Effect of Buffer Strength

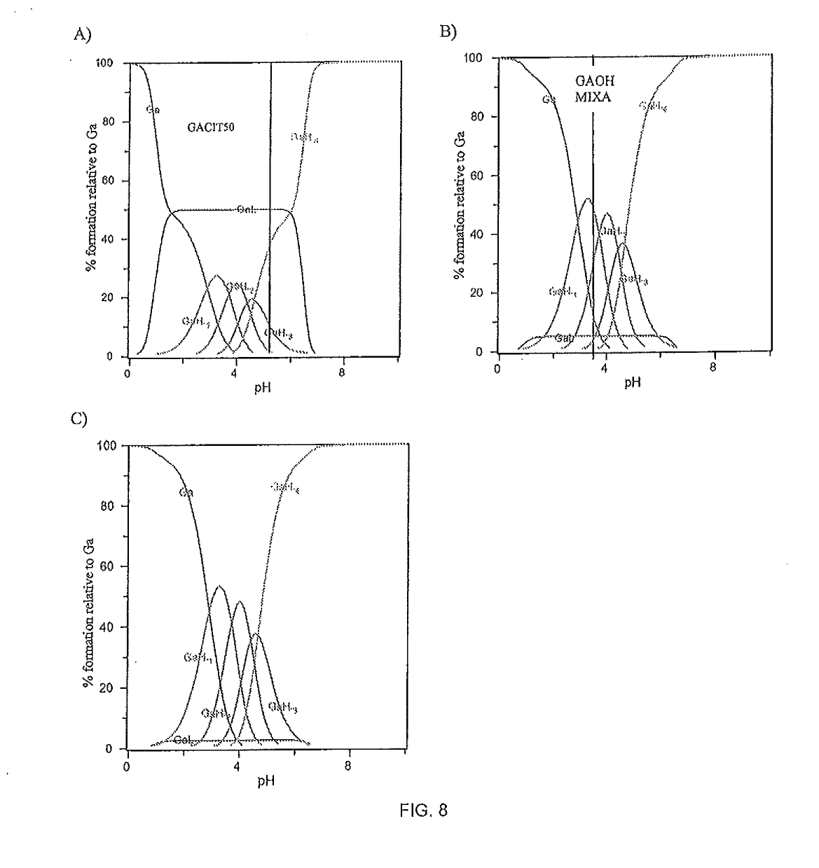

Between pH 2 and 6, using a saturated gallium concentration in a 1 M citrate buffer generated 50% gallium citrate and a mixture of hydroxide complexes, with the number of hydroxide ligands associated increasing with pH (FIG. 8A). Decreasing the buffer strength 10-fold had the effect of increasing the proportion of gallium hydroxide species present within this range. Between pH 2 and 4, saturated gallium in 0.1 M citrate was mostly present (>80%) as a combination of Ga(OH)²⁺ and Ga(OH)₂¹⁺. The relative proportion of Ga(OH)₂⁺¹ to Ga(OH)⁺² increased with pH (FIG. 8B), however above pH 4 Ga(OH)₃ and Ga(OH)₄¹⁻ became increasingly prevalent. Reducing the buffer strength further to 0.05 M citrate had little effect on the Ga speciation. It should be noted that using a saturated gallium solution, a maximum formation of 50% gallium citrate was obtained (FIG. 8C).

(iii) Gallium Species Optimization

Based on the findings above, different combinations of pH, buffer strength and gallium concentration were selected to enable the formation of six specific combinations of gallium species to determine the effect of gallium complexation on skin permeation (Table 8). The relative abundance of Ga species present in each system was determined using HYSS speciation plots described above and the overall charge of the species present was tabulated. The formulations designated “GACIT100” and “GA100” in Table 2below contain gallium only present as negatively charged gallium citrate complexes and positively charged gallium ions, respectively. “GACIT50,” “GAOHMIXA,” “GAOHMIXB,” and “GAOHMIXC” in Table 8 all contain different combinations of all the species present in the speciation plots and have been grouped together as the “mixed hydroxides.”

TABLE 8
Theoretical absolute Ga concentration (mg/ml) for each gallium donor
species generated using HYSS speciation plots produced to investigate the effect of
speciation on gallium deposition in the skin.

| Donor species | | | | | | |
|---|---|---|---|---|---|---|
| reference | | | | | | |
| (buffer strength, pH) | Ga³⁺ | Ga citrate | GaOH²⁺ | Ga(OH)₂⁺ | Ga(OH)₃ | Ga(OH)₄⁻ |
| GACIT100 | — | 1.5 | — | — | — | — |
| (0.05M, 4.10) | | | | | | |
| GA100 | 1.5 | — | — | — | — | — |
| (0.001M, 2.03) | | | | | | |
| GACIT50 | — | 83.35 | — | — | 16.67 | 66.68 |
| (1.00M, 5.21) | | | | | | |
| GAOHMIXA | 16.67 | 8.34 | 83.35 | 50.01 | 8.34 | — |
| (0.10M, 3.83) | | | | | | |
| GAOHMIXB | — | 4.17 | — | — | 1.67 | 10.84 |
| (0.05M, 5.63) | | | | | | |
| GAOMIXC | — | 8.34 | 16.67 | 66.68 | 50.01 | 25.01 |
| (0.05M, 4.30) | | | | | | |

### (iv) Effect of Speciation and ITP on Gallium Permeation

Passive Ga permeation across full thickness human skin from the GA100 donor species was more than four times greater than from the other donor species. After 65 h of passive permeation 28±5 μg/cm² of elemental Ga was detected in the receiver fluid (Table 9), whereas less than 7 μg/cm² elemental Ga had permeated the skin from all the other donor solutions. The rate of passive permeation from all the other donors were statistically similar (P>0.05 ANOVA) (Table 9). The passive data for the GACIT100 donor has not been performed but will need to be evaluated in the future.

The application of 60 min of anodal ITP to the GA100 donor solution increased gallium permeation across the skin over 65 h by 29% over passive delivery, from 28±5 to 36±6 μg/cm². This increase was not statistically significant due to the variability in the skin data (P>0.05, ANOVA). The application of 60 min anodal ITP to the GACIT100 donor solution did not appear to affect gallium permeation, assuming that the passive permeation from this system is similar to the other donor solutions. Applying cathodal ITP to the GACIT100 donor significantly increased the amount of Ga detected in the receiver from 4±8 to 298±331 μg/cm², an approximate increase of 7350% compared to anodal ITP. A similar increase is expected to be found compared with passive once the experiment is performed. In addition, the amount of gallium detected in the receiver fluid of the cells was increased by both cathodal and anodal ITP for all the mixed hydroxides (GACIT50, GAOHMIXC and GAOHMIXA) compared to passive permeation alone (Table 9). The observed increase in flux using anodal ITP was greatest for the GAOHMIXA donor. The Ga detected from this donor solution increased from 3±8 μg/cm² to 2397±860 μg/cm², an increase in flux of approximately 80,000% compared to passive. Detection of elemental Ga in the receiver fluid from the GACIT50 donor was increased from 7±12.7 μg/cm² to 2032±2160 μg/cm² by the application of anodal ITP, an increase in gallium permeation of approx. 29,000% compared to passive. Detection in the receiver fluid from the GAOHMIXC donor was increased from 4±16 μg/cm² to 63±22 μg/cm² by anodal ITP, an increase in gallium permeation of approx. 1500% compared to passive (Table 9). The enhancement of Ga permeation from the mixed hydroxide donors observed using cathodal ITP was less marked than with anodal ITP, but was still statistically significant for both GACIT50 and GAOHMIXA (ANOVA P<0.05) (Table 9). The observed increase in Ga detection using cathodal ITP for the GACIT50 donor was from 7±13 μg/cm² to 150±159 μg/cm², an approx. increase in flux of 2040% compared to passive. This was the highest cathodal increase from all the mixed hydroxide donors, but was still only half the amount detected from the GACIT100 donor. Detection of elemental Ga in the receiver fluid from the GAOHMIXA donor was increased from 3±8 μg/cm² to 52±48 μg/cm² by the application of cathodal ITP, an increase in gallium permeation of approx. 1600% compared to passive (Table 9),

In summary, the highest level of Ga delivery across the skin using anodal ITP was seen with GAOHMIXA, which contains the positively charged GaOH²⁺ and Ga(OH)⁺ as the predominant species (83.35 and 50.01 mg/mL respectively). The highest level of Ga delivery across the skin using cathodal ITP was seen with GACIT100, which contains the potentially negatively charged citrate complex as the predominant species (1.5 mgmL⁻¹).

TABLE 9
Effect of gallium speciation and ITP upon gallium permeation across full
thickness human skin obtained using upright Franz cells (n = 6-8).
Each cell subjected to 60 min of 615 μA/cm² ITP, where
appropriate in either an anodal or cathodal orientation and left for
65 h in a thermostatically controlled water bath at 37° C.
Receiver fluid samples were assayed for total gallium content using the ion
exchange LC assay ± 1s.d.

| | Passive Ga | Anodal ITP Ga | Cathodal ITP Ga |
|---|---|---|---|
| Ga donor | permeation | permeation | permeation |
| solution | (μg/cm²) | (μg/cm²) | (μg/cm²) |
| GA100 | 28 ± 5 | 36 ± 6 | — |
| GACIT100 | — | 4 ± 8 | 298 ± 331 |
| GACIT50 | 7 ± 12 | 2032 ± 2160 | 150 ± 159 |
| GAOHMIXC | 4 ± 16 | 63 ± 22 | — |
| GAOHMIXA | 3 ± 8 | 2397 ± 860 | 52 ± 48 |

(v) Effect of ITP and Speciation on Gallium Deposition within the Skin

The application of 60 minutes of anodal ITP to the GAOHMIXA donor system significantly increased the elemental gallium skin deposition from 0.288±0.26 mg/cm² to 1.203±0.25 mg/cm² (p<0.05, ANOVA) (Table 10). A further increase in gallium deposition was observed when the cells were kept in contact with the donor solution for an additional 23 hrs post ITP application (1.888±1.16 mg/cm²). A similar pattern of skin deposition was observed with cathodal ITP delivery from the GACIT100 donor. Analysis of the skin immediately after the application of 60 min of cathodal ITP showed a deposition of 1.281±0.27 mg/cm² of elemental gallium in the skin. The level of Ga deposition increased to 1.550±0.35 mg/cm² when the cells were kept in contact with the donor solution for an additional 23 hrs post ITP application (Table 10). However as no passive control data is yet available for comparison for the gallium citrate donor, the statistical significance of these results relative to the passive control cannot be assessed. As with the gallium permeation study, the passive gallium deposition from the GACIT100 donor will be completed in the future. However it is assumed that the passive deposition is of a similar level to that of the GAOHMIXA donor.

The gallium skin deposition levels after one hour of anodal (GAOHMIXA) and cathodal (GACIT100) ITP were statistically the same from both of the Ga donor solutions (ANOVA, P>0.05). A higher degree of Ga deposition was observed after 24 h of permeation from the GAOHMIXA donor solution compared to GACIT100, although this difference was not statistically significant due to the variability in the human skin data (p>0.05, ANOVA). It should also be recognised that the total applied Ga concentration was greater with the mixed gallium hydroxide donor compared to the gallium citrate system, only 1.5 mg/mL of the citrate complex was applied in the GACIT100 donor, whereas a combined concentration of 333.36 mg/mL of positively charged GaOH complexes were applied in the GAOHMIXA donor solution.

TABLE 10
Effect of gallium speciation and ITP upon gallium deposition into full
thickness human skin obtained using upright Franz cells (n = 6-8).
Each cell was subjected to 60 min of 615 μA/cm² ITP,
where appropriate in either an anodal or cathodal orientation and
left for 0 or 24 h in a thermostatically controlled water bath
at 37° C. Cells were sacrificed and the skin homogenised
and filtered to measure the gallium deposition.

| | | | ITP Ga skin | ITP Ga skin |
|---|---|---|---|---|
| | | 60 min passive | deposition | deposition |
| Ga donor | ITP | skin deposition | 60 min | 24 h |
| solution | orientation | (mg/cm²) | (mg/cm²) | (mg/cm²) |
| GAOHMIXA | Anodal | 0.288 ± 0.26 | 1.203 ± 0.25 | 1.888 ± 1.16 |
| GACIT100 | Cathodal | — | 1.281 ± 0.27 | 1.550 ± 0.35 |

### D. Discussion

The pH of human skin is generally thought to have a value of around 5.2, due to the acid mantle which is a thin oily layer on the skin surface. However, many factors have been identified which can influence skin pH including age, anatomical site, skin moisture and topical applications such as detergents (Schmid-Wendter & Korting, 2006). Healthy skin possesses an iso-electric point of between pH 3 and 4. Below this pH the skin will have a net positive charge; above tins pH the skin will display net negative charge. To be able to investigate the effect of Ga speciation on skin permeation, it was important to demonstrate that the pH of the solution in contact with the skin did not affect permeation. It was demonstrated that between pH 2 and 5 the permeation across the skin of water, a small polar molecule, was not affected by pH. Therefore, it was possible to directly compare the effect of Ga speciation within this pH range with the knowledge that any differences in permeation observed are not due to the pH of the solution.

Gallium is a strong acid and is stable as free, positively charged irons in acidic aqueous conditions below pH 2. Generally, as the basicity of the conditions increased, gallium bound increasingly to the hydroxyl groups predominant in the solution to form GaOH²⁺, Ga(OH)₂⁺, the uncharged Ga(OH)₃ and the negatively charged Ga(OH)₄⁻ species. In citrate buffered solutions of between pH 2 and 6, gallium could also bind with citric acid molecules hi solution to form gallium citrate, which may be either neutral or negatively charged. It is assumed here that gallium and citrate forms a 1:1 complex with an overall net negative charge, however the actual structure is yet to be elucidated and may form many differing molecular structures depending on d-orbital involvement. Increasing the Ga concentration in the solution had the effect of decreasing the relative proportion of gallium citrate present as the ratio of gallium ions to citrate ions increases and there were not enough citrate ions present to compete with the smaller hydroxyl groups for the free gallium bonding orbitals. The same pattern was observed when the buffer strength was reduced as the total citrate ion content is reduced and can no longer compete with the hydroxyl groups present in the system.

To investigate the effect of speciation on gallium permeation, donor solution conditions were carefully selected from the speciation plots to obtain conditions where gallium was present as 100% positively charged free gallium ions, 100% citrate complex (neutral or negatively charged) or as a combination of positively charged mixed gallium hydroxide complexes. From these donor solution complexes it was possible to investigate the effect of the gallium complex on both passive and iontophoretic full thickness skin permeation.

The species of gallium present in the donor solution had a significant effect on the passive permeation of elemental gallium across the skin. Gallium citrate and gallium hydroxide complexes both permeated the skin at a similar rate, but free gallium ions passively penetrated the skin at a greater rate, possibly due to their smaller molecular size and reduced steric burden. However, free ions are only present at pH 2 or below and these acidic conditions may have local tolerability issues. Also, these species will be very reactive once they enter the local pH of the skin cells and interstitial fluid.

The application of ITP enhanced the permeation of gallium across full thickness human skin. The extent of this enhancement was determined by the species of gallium present in the donor solution applied to the skin sample, as this determines the ITP mechanism which was prevalent i.e. electrorepulsion, electropertubation or electroosmosis (Hostynek, 2003). Anodal ITP primarily enhanced gallium delivery from systems containing high concentrations of the positively charged gallium hydroxide complexes. This was thought to be due to the presence of the small and positively charged Ga(OH)⁺⁺ species and to a lesser extent the Ga(OH)₂⁺ species. The application of a positive charge to these complexes would result in electro repulsion and enhanced permeation across the SC. The GAOHMIXA donor solution contains the highest concentration of these complexes and, as expected, displays the greatest amount of Ga permeation after the application of anodal ITP. However, the relationship between anodal ITP enhanced gallium permeation from these solutions and the species present is not simple. The buffer strength appears to have a marked effect on the gallium permeability across the skin. As iontophoretic permeation is a competitive process, the concentration of competing ions will affect the rate for each individual species. Although it was possible to calculate the initial concentration of the competing ions in the donor solutions, other ions such as Na⁺, OH⁻, H⁺, Cl⁻ and K⁺ from the skin sample may swamp the ability of the gallium to carry the charge and therefore the transport number observed. It was not possible to keep the ratio of gallium ions and citrate ions constant throughout all the solutions as it is a dynamic system and, as a result, differing degrees of competition for the ITP applied would exist between the systems. The observed increase in gallium permeation appears to be less marked if significant amounts of the neutral complex Ga(OH)₃ are present. This pattern was not observed for Ga delivery from the GACIT50 donor solution, which contained high levels of Ga(OH)₃ and was enhanced by both anodal and cathodal ITP application. This non-charge specific permeation enhancement suggests that an electroosmotic mechanism may exist for the delivery of the neutral species, however the exact mechanism has not been elucidated. Another possible explanation for this is the decreased solubility and stability in acidic conditions of the amorphous Ga(OH)₃ species (Kulprathipanja & Hnatowich, 1977; Wood & Samson, 2006). Precipitation of this species would be hard to identify once applied to the skin in a Franz cell, however its presence would affect the ability of the other ions to permeate into the skin possibly impairing permeation by competition.

Cathodal ITP appeared to primarily enhance gallium delivery from the systems containing gallium citrate, it may be necessary to test this theory by assessing the cathodal delivery of the GA100 donor mixture, which was the only solution to contain no citrate complex. This suggested that the gallium citrate complex had a net negative charge in these conditions and was being pushed across the skin by electrorepulsion. GACIT100 displayed the most efficient Ga permeation using cathodal delivery although it did not possess the greatest concentration of citrate complex in the donor. The other donor solutions contained mixtures of Ga complexes and higher buffer strengths which would have increased competition within the system in the same way as for anodal ITP. The fact that less gallium was being detected from cathodal delivery compared to anodal delivery may be due to the increased molecular size of gallium citrate (259-637 Da) compared to the gallium hydroxide complexes (86.72-120,72 Da). Molecules with a mass greater than 500 Da possess greater steric burden within the skin and therefore slower rates of permeation (Barry, 1991).

Elemental gallium deposition within the skin was enhanced by anodal and cathodal ITP delivery of the mixed hydroxide and gallium citrate systems respectively. The effect was observed immediately after the application of 60 min of ITP and was greatest after 24 h of additional passive permeation time. This suggests that not only are the positive Ga(OH)²⁺ and Ga(OH)₂⁺ and the negative gallium citrate species being immediately forced into the skin by electrorepulsion, but the effectiveness of the skin to act as a barrier to permeation remains greatly reduced even after the removal of the ITP electrodes allowing enhanced drug permeation to continue. This reduction in skin impedance caused by the application of an electrical charge such as with ITP is known to continue for some time after the removal of the iontophoretic device (Kalia & Guy, 1995). In addition, the level of Ga deposition achieved in the skin from the GAOHMIXA and GACIT100 donor solutions after ITP was far greater than the suggested effective concentration ranges of 0.25-100 uM gallium nitrate and 1-1000 ng gallium nitrate per mg dry weight tissue (Goncalves et al., 2002). The location of the Ga deposition within the skin is still unknown as only the surface adhered gallium was removed by tape stripping, thus the SC remained intact prior to homogenisation and assay. Further investigation is required to determine if the gallium is present at these concentrations in the dermal collagen target site.

The data presented here suggest that uncompleted gallium ions can passively penetrate the stratum corneum more readily than some of the Ga complexes, possibly due to their small molecular size. Gallium citrate and gallium hydroxide are subject to a greater degree of steric burden due to their larger molecular weights. The permeation of gallium citrate (−ve) through the skin is enhanced by the application of cathodal ITP, resulting in greater elemental gallium present in the skin compared to passive permeation alone. The permeation of Ga(OH)₂⁺ and Ga(OH)²⁺ through the skin is enhanced by the application of anodal ITP resulting in greater elemental gallium present in the skin compared to passive permeation alone. The presence of hydroxide ions in the system dominates the ITP delivery due to their high charge density and relatively small molecular weight compared to the gallium citrate complex, Ga(OH)₃ and Ga(OH)₄⁻. The presence of Ga(OH)²⁺ in the system results in the most efficient anodal ITP delivery and greatest increase in elemental gallium deposition within the skin compared to passive permeation alone.

### REFERENCES

Alderighi, L., Gans, P., Ienco, A., Peters, D., Sabatini, A., & Vacca, A. 1999, “Hyperquad simulation and speciation (HySS): a utility program for the investigation of equilibria involving soluble and partially soluble species”, Coordination Chemistry Reviews, vol. 184, no. 1, pp. 311-318.
Barry, B. W. 1991, “Modern methods of promoting drug absorption through the skin”, Molecular Aspects of Medicine, vol. 12, no. 3, pp. 195-241.
Benezeth, P., Diakonov, I. I., Pokrovski, G. S., Dandurand, J. L., Schott, J., & Khodakovsky, I. L. 1997, “Gallium speciation in aqueous solution. Experimental study and modelling: Part 2. Solubility of [alpha]-GaOOH in acidic solutions from 150 to 250[degree sign]C. and hydrolysis constants of gallium (III) to 300[degree sign]C.”, Geochimica et Cosmochimica Acta, vol. 61, no. 7, pp. 1345-1357.
Bernstein, L. R. 1998, “Mechanisms of Therapeutic Activity for Gallium”, Pharmacological Reviews, vol. 50, no. 4, pp. 665-682.
Bockman, R. S., Guidon, P., Pan, L. C, Salvatori, R., & Kawaguchi, A. 1993, “Gallium nitrate increases type I collagen and fibronectin mRNA and collagen protein levels in bone and fibroblast cells”, Journal of Cellular Biochemistry no. 52, pp. 396-403.
Briggs, S. L. 2005, “The role of fibronectin in fibroblast migration during tissue repair, [Review] [38 refs]”, Journal of Wound Care.14(6);284-7.
Fullerton, A. & Hoelgaard, A. 1988, “Binding of nickel to human epidemis in vitro”, British journal of dermatology, vol. 119, pp. 675-682.
Goncalves, J., Wasif, N., Esposito, D., Coico, J. M., Schwartz, B., Higgins, P. J., Bockman, R. S., & Staiano-Coico, L. 2002, “Gallium nitrate accelerates partial thickness wound repair and alters keratinocyte integrin expression to favor a motile phenotype”, Journal of Surgical Research, vol. 103, no. 2, pp. 134-140.
Harris, W, R. & Martell, A. E. 1976, “Aqueous complexes of gallium (III)”, Inorganic chemistry, vol. 15, no. 713.
Higuchi, T. 1959, “Physical Chemical Analysis of Percutaneous Absorption process from creams and ointments”, Journal of the Society of Cosmetic Chemists, no. 11, pp. 85-97.
Hostynek, J. J. 2003, “Factors determining percutaneous metal absorption”, Food and Chemical Toxicology, vol. 41, no. 3, pp. 327-345.
Hostynek, J. J., Hinz, R. S., Lorence, C. R., Price, M. & Guy, R. H. 1993, “Metals and the skin”, Critical reviews in toxicology, vol. 23, no. 2, pp. 173-235.
Kalia, Y. N. & Guy, R. H. 1995, “The Electrical Characteristics of Human Skin In-Vivo”, Pharmaceutical Research, vol. 12, no. 11, pp. 1605-1613.
Kulprathipanja, S. & Hnatowich, D. J. 1977, “A method for determining the pH stability range of gallium radiopharmaceuticals”, The International Journal of Applied Radiation and Isotopes, vol. 28, no. 1-2, pp. 229-233.
Maddock, W. G. & Coller, F. A. 1933, “The role of the extremities in the dissipation of heat”, american journal of physiology no. 106, pp. 589-596.
Nazarenko, V., Antonovich, V., & Nevskaya, E. 1968, “Spectrophotometric determination of the hydrolysis constants of gallium ions”, Russian journal of Inorganic chemistry, vol. 13, no. 6, p. 1574,
Schmid-Wendter, M. H. & Korting, H. C. 2006, “The pH of the skin surface and its impact on the barrier function”, Skin Pharmacology and Physiology no. 19, pp. 296-302.
Wood, S. A. & Samson, I. M. 2006, “The aqueous geochemistry of gallium, germanium, indium and scandium”, Ore Geology Reviews, vol. 28, no. 1, pp. 57-102.

### Example 6

### Iontophoretic Delivery of Gallium into Full Thickness Human Skin In Vitro

Gallium (Ga) has been reported to have a number of biological effects, including stimulation of type 1 collagen and fibronectin synthesis and acceleration of wound healing. Delivery of this agent into skin may be challenging as it forms different coordination complexes depending on pH, buffer, and concentration. The present study explored using iontophoresis to entrance the intradermal delivery of gallium.

Anodal and cathodal iontophoresis (0.3 mA/cm² for 15 min) were carried out using full thickness human cadaver skin in Franz diffusion cells. Ga nitrate solutions (0.15% in 0.05M citrate buffer, or a saturated solution of 16.6% in 0.1M/1M citrate buffer) at different pH conditions (2 to 8) were evaluated. The amount of gallium permeated through and deposited into the skin was measured using receptor analysis and skin extraction, respectively.

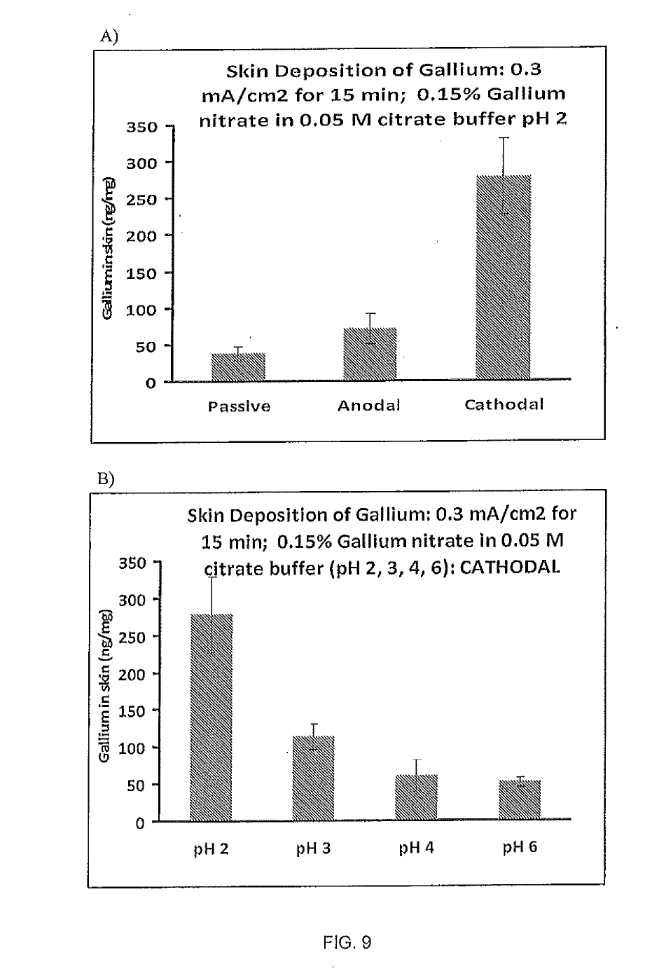

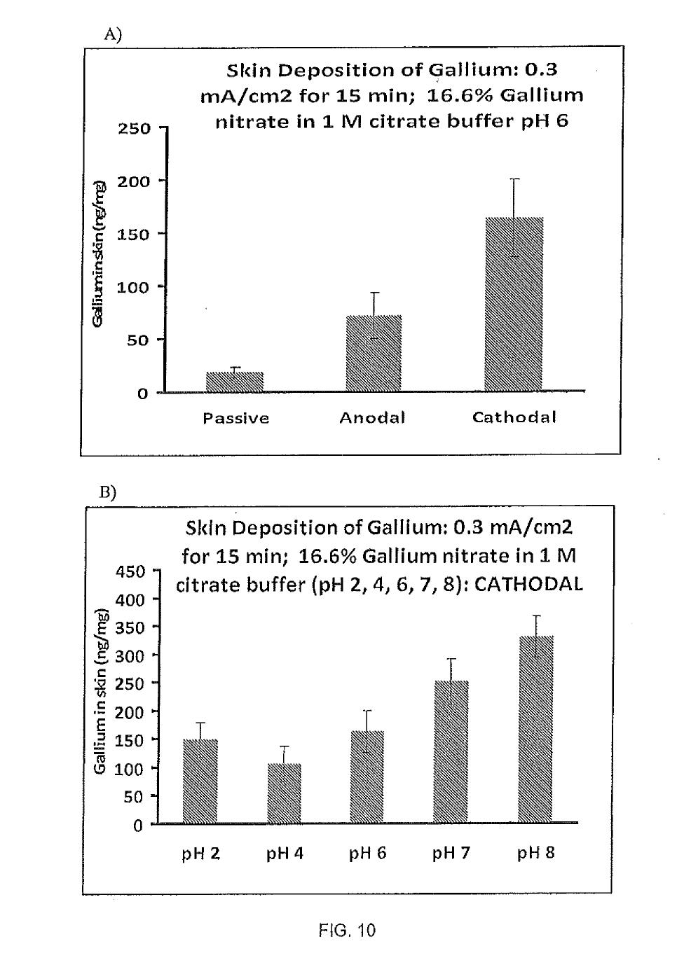

Ga was not detected in the receptor for any of the conditions tested. Cathodal iontophoresis significantly enhanced the delivery into the skin (relative to anodal/passive) of 0.15% gallium nitrate solution in 0.05M citrate buffer at all three pH values [277.90±51.24 ng/mg (pH 2), 113.16±17.65 ng/mg (pH 3), 60.5±21.11 ng/mg (pH 4) and 51.16±6.65 ng/mg (pH 6)] (FIGS. 9A and 9B). According to the HYSS speciation plots described in Example 5, the expected species for 0.15% gallium nitrate in 0.05M citrate buffer at pH 2 (FIG. 9A) would be >95% gallium citrate (−1 charge) and <5% Ga (+3 charge) with a net negative charge and therefore would favor cathodal iontophoresis. At the 0.1 M buffer strength (data not shown), anodal iontophoresis enhanced delivery of the 16.6% Ga nitrate solution (relative to cathodal/passive) at the lower pH values (2 & 4; 91.74±13.09 ng/mg and 74.42±22.31 ng/mg, respectively), while at pH 6 the skin levels were found to be comparable between anodal and cathodal delivery (62.19±23.76 ng/mg and 99.62±36.44 ng/mg, respectively) but greater than passive. At the higher buffer strength (1M), skin deposition was comparable at the lower pH values (2 & 4) between anodal and cathodal iontophoresis, but cathodal iontophoresis was prominent at pH 6 (163.72±36.72 ng/mg) relative to anodal iontophoresis and passive (FIG. 10A) and was further enhanced at pH 7 (250.49±39.76 ng/mg) and pH 8 (329.52±36.10 ng/mg) (FIG. 10B). The expected species for 16.6% gallium nitrate in 1M citrate buffer at pH 6 (FIG. 10A) would be 50% gallium citrate (−1 charge) and 50% Ga(OH)₄ (−1 charge) with a net negative charge and therefore would favor cathodal iontophoresis. The amount delivered into the skin for passive delivery was significantly lower in all cases (10.96-22.34 ng/mg).

The data demonstrate the utility of iontophoresis in enhancing the skin delivery of gallium. The wide difference in Ga delivery with the change in current polarity, pH, Ga concentration, and buffer strength is most likely due to the presence of different gallium species and coordination complexes, which may have different charges, at the various experimental conditions tested.

### Example 7

### Development of a Stable Gel Formulation of Gallium

Based on the requirements for a stable, aqueous gel formulation which meets the requirements of iontophoretic delivery (which are not necessarily equivalent to what Is typically required for a standard topical formulation), the formulation may include:
(1) Charged gallium ion (III) in an amount 0.1 to 20% w/w (using 16.6% gallium nitrate in 1M citrate at pH 8, maximum skin deposition by iontophoretic drug delivery at 0.3 mA/cm² for 15 min was 78 ug/cm², which is 0.78 mg/ml or 30× higher than maximum effective dose of 100 uM or 0.0256 mg/ml; Alternatively, maximum drug delivery was 330 ng/mg, which is in range of 1-1000 ng/mg effective dose range as specified in U.S. Pat. No. 6,365,514 issued to Bockman et al.);
(2) A suitable buffer system, preferably citrate and/or phosphate sufficient to control pH between 6.5-7.5 or alternative buffers including glutamate, aspartate, ascorbate, tartrate, malate, fumarate, edentate, gluconate, or succinate to control pH between 3-4;
(3) A suitable thickener, preferably hydroxyethyl cellulose or polyvinyl pyrrolidone to build sufficient rheology of the formulation to create a single-phase, low viscosity gel;
(4) Optionally, a suitable stabilizer, chelator (e.g., ascorbate) and antioxidant (preferably ascorbic acid or tocopherol/vitamin E);
(5) Optionally, a suitable agent (preferably a saturated fatty acid such as isostearic acid or palmitic acid, or TPGS) which can increase residence time and build a depot effect;
(6) Optionally, a suitable preservative, preferably benzoic acid in an amount (0.01-0.1% w/w);
(7) Optionally, a suitable solubilizing agent(s), preferably glycerin (30-50%);
(8) Optionally, a permeation enhancer (preferably Transcutol P, a transporter such as aspartic acid/gluconic acid, or chitosan or cyclodextrin);
(9) Optionally, an emollient (preferably glycerin) in an amount of 1-30% w/w.

While this invention has been particularly shown and described with references to preferred embodiments thereof, it will be understood by those skilled in the art that various changes in form and details may be made therein without departing from the scope of the invention encompassed by the appended claims.

What is claimed is:

1. A formulation suitable for iontophoresis comprising gallium nitrate in a buffer wherein the buffer has an ionic strength from about 0.01 to about 1 M and wherein the formulation comprises a gallium species selected from the group consisting of gallium citrate species and gallium hydroxide species.

2. The formulation of claim 1, wherein the buffer is a citrate buffer,

3. The formulation of claim 2, wherein the buffer has an ionic strength from about 0.05 M to about 0.10 M.

4. The formulation of claim 1, wherein the formulation has a pH from about 2 to about 12.

5. The formulation of claim 2, wherein the formulation has a pH from about 2 to about 12.

6. The formulation of claim 5, wherein the formulation has a pH from about 3 to about 4.

7. The formulation of claim 5, wherein the formulation has a pH from about 6 to about 12.

8. The formulation of claim 5, wherein the formulation has a pH from about 7 to about 8.

9. The formulation of claim 2, wherein the formulation comprises from about 0.1 to about 20% w/v gallium nitrate.

10. The formulation of claim 9, wherein the formulation comprises from about 15% to about 20% w/v gallium nitrate.

11. The formulation of claim 10, wherein the formulation comprises from about 16 to about 17% w/v gallium nitrate.

12. The formulation of claim 11, wherein the formulation comprises about 16.7% w/v gallium nitrate.

13. The formulation of claim 12, wherein the formulation has a pH from about 7 to about 8 and wherein the buffer has ionic strength of about 1 M.

14. A formulation suitable for cathodal iontophoresis comprising gallium nitrate in a citrate buffer wherein the buffer has an ionic strength of about 0.05 M and wherein the formulation has a pH from about 3.8 to about 4.2.

15. The formulation of claim 14, wherein the pH is about 4.

16. A formulation suitable for anodal iontophoresis comprising gallium nitrate in a citrate buffer wherein the buffer has an ionic strength of about 0.10 M and a pH from about 3.5 to about 4.

17. The formulation of claim 16, wherein the pH is about 3.8.

18. The formulation of claim 16, wherein the formulation comprises from about 15% to about 20% w/v gallium nitrate.

19. A formulation suitable for iontophoresis comprising gallium nitrate in a citrate buffer wherein the gallium species in the formulation comprises gallium citrate species.

20. The formulation of claim 19, wherein the gallium species consists of gallium citrate species.

21. The formulation of claim 19, further comprising gallium hydroxide species.

22. The formulation of claim 19, wherein the gallium hydroxide species are positively charged gallium hydroxide species.

23. The formulation of claim 21, wherein the gallium hydroxide species are selected from the group consisting of Ga(OH)₂⁺, Ga(OH)²⁺, Ga(OH)₄ and Ga(OH)₄⁻.

24. The formulation of claim 23, wherein the gallium species consist of free gallium cations, gallium citrate, Ga(OH)₂⁺, Ga(OH)²⁺ and Ga(OH)₃.

25. A method of administering gallium to a patient in need thereof comprising iontophoretically administering to the skin of said patient the formulation of claim 1.

26. The method of claim 25, wherein a current density of at least about 10 uA/cm² is applied.

27. The method of claim 25, wherein a current density from about 200 uA/cm² to about 500 uA/cm² is applied.

28. The method of claim 25, wherein current is applied for a time greater than about 5 minutes.

29. The method of claim 28, wherein the current is applied for a time greater than about 15 minutes.

30. The method of claim 29, wherein the current is applied for a time greater than about 30 minutes.

31. The method of claim 25, wherein iontophoresis is applied at about 0.1 mA*mm to about 100 mA*min.

32. A method of administering gallium to a patient in need thereof comprising iontophoretically administering to the skin of said patient the formulation of claim 21.

33. The method of claim 32, wherein a current density of at least about 10 uA/cm² is applied.

34. The method of claim 32, wherein current is applied for a time greater than about 60 minutes.

35. A method of upregulating the synthesis of collagen or fibronectin in the skin in a patient in need thereof comprising iontophoretically administering to the skin of the patient the formulation of claim 1.

36. The method of claim 35, wherein the upregulation of collagen or fibronectin results in increased thickness or elasticity in the skin of the patient.
